(19) United States
(12) Patent Application Publication
(10) Pub. No.: US 2005/0244498 A1
(43) Pub. Date: Nov. 3, 2005

(54) Modified-release compositions of at least one form of venlafaxine

(75) Inventors: Pawan Seth, Irvine, CA (US); Paul J. Maes, Toronto (CA)

Correspondence Address: CROWELL & MORING LLP;INTELLECTUAL PROPERTY GROUP, P.O. BOX 14300, WASHINGTON, DC, 20044-4300, US

(73) Assignee: Biovail Laboratories, Inc., Collymore Rock (BB)

(21) Appl. No.: 11/003,028

(22) Filed: Dec. 3, 2004

Related U.S. Application Data
(63) Continuation-in-part of application No. 10/244,059, filed on Sep. 13, 2002.
Application No. 10/244,059 is a continuation-in-part of application No. 09/953,101, filed on Sep. 14, 2001, now abandoned.

(51) Int. Cl. A61K 31/277; A61K 9/00; A61K 9/24

(52) U.S. Cl. 424/472; 514/522

(57) ABSTRACT

The present invention relates to a modified release composition of at least one form of venlafaxine, which is an enhanced absorption delayed controlled release composition for oral administration suitable for once daily dosing. The composition comprises a core comprising at least one form of venlafaxine selected from the group consisting of venlafaxine, an active metabolite of venlafaxine, a pharmaceutically acceptable salt of venlafaxine, a pharmaceutically acceptable salt of an active metabolite of venlafaxine, and combinations thereof, and a pharmaceutically acceptable excipient. The composition further comprises a modified release coating which substantially surrounds the core. The compositions of the invention provide enhanced absorption delayed controlled release of the at least one form of venlafaxine such that the combined geometric mean ratio of the composition of the invention to the reference product for the AUC₀₋ₜ or the Cₘₐₓ for venlafaxine and its active metabolite O-desmethylvenlafaxine is greater than 2 after first administration of the composition under fed or fasting conditions.

### RELATED APPLICATIONS

This application is a Continuation-In-Part (CIP) of U.S. Ser. No. 10/244,059, filed Sep. 13, 2002, which is in turn a CIP of U.S. Ser. No. 09/953,101 filed Sep. 14, 2001, now abandoned. Both applications are incorporated herein by reference in their entirety.

### FIELD OF THE INVENTION

The present invention relates to modified release compositions for oral administration of at least one form of venlafaxin, to processes for their preparation and to their medical use. In particular, the modified release composition relates to an enhanced absorption delayed controlled release composition of at least one form of venlafaxin.

### BACKGROUND OF THE INVENTION

An ideal dosage regimen for many medications is that by which an acceptable therapeutic concentration of drug at the site(s) of action is attained immediately and is then maintained constant for the duration of the treatment. Providing dose size and frequency of administration are correct, therapeutic “steady-state” plasma concentrations of a drug can be achieved promptly and maintained by the repetitive administration of conventional peroral dosage forms. However, there are a number of potential limitations associated with conventional peroral dosage forms. These limitations have led pharmaceutical scientists to consider presenting therapeutically active molecules in “extended-release” preparations.

Oral ingestion is the traditionally preferred route of drug administration, providing a convenient method of effectively achieving both local and systemic effects. An ideal oral drug delivery system should steadily deliver a measurable and reproducible amount of drug to the target site over a prolonged period. Extended-release (ER) delivery systems provide a uniform concentration/amount of the drug at the absorption site and thus, after absorption, allow maintenance of plasma concentrations within a therapeutic range over an extended period of time, which can minimize side effects and also reduces the frequency of administration. ER dosage forms release drug slowly, so that plasma concentrations are maintained at a therapeutic level for a prolonged period of time. Typically, these products provide numerous benefits compared with immediate-release compositions, including greater effectiveness in the treatment of chronic conditions, reduced side effects, greater convenience, and higher levels of patient compliance due to a simplified dosing schedule. Because of the above advantages, such systems form a major segment of the drug delivery market.

Many drug delivery systems have been developed with the aim of eliminating the cyclical changes in plasma drug concentration seen after the administration of a conventional delivery system. A variety of terms have been used to describe these systems: delayed release, repeat action, prolonged release, sustained release, extended release, controlled release and modified release. It is interesting to note that the USP considers that the terms controlled release, prolonged release, sustained release and extended-release are interchangeable.

Controlled-release formulations have been described in the prior art and many methods have been used to provide controlled-release pharmaceutical dosage forms in order to maintain therapeutic serum levels of medicaments and to minimize the effects of missed doses of drugs caused by a lack of patient compliance. Anti-depressants are excellent candidates for controlled-release formulations as discontinuation of these drugs, most often as a result of a lack of patient compliance due to a complicated or multiple daily dosing schedule, can often result in severe discontinuation symptoms.

Venlafaxine, chemically designated as (R/S)-1-[2-(dimethylamino)-1-(4methoxyphenyl)ethyl]cyclohexanol or (±)-1-[a [α-(dimethylamino)methyl]p-methoxybenzyl]cyclohexanol, is a bicyclic compound with antidepressant properties affecting chemical messengers within the brain. These chemical messengers, called neurotransmitters, can for example be serotonin, dopamine, and norepinephrine. Neurotransmitters are manufactured and released by nerve cells. The neurotransmitters travel to neighboring nerve cells and cause the cells to become more or less active. It is believed that an imbalance in these neurotransmitters is the cause of depression and also may play a role in anxiety. Venlafaxine is believed to work by inhibiting the release or affecting the action of these neurotransmitters.

Venlafaxine is chemically unrelated to other antidepressants, but is sometimes categorized as a serotonin-norepinephrine reuptake inhibitor (SNRI). At low dosages, venlafaxine blocks serotonin reuptake, similarly to a selective serotonin reuptake inhibitor (SSRI). At medium dosages, venlafaxine blocks the reuptake of norepinephrine as well as serotonin. At high dosages, venlafaxine blocks the reuptake of norepinephrine, serotonin and is also a weak blocker of the reuptake of dopamine.

Venlafaxine is well absorbed after oral administration and its metabolism has been well documented. Following absorption, venlafaxine undergoes extensive pre-systemic metabolism in the liver, primarily to O-desmethylvenlafaxine (ODV), but also to N-desmethylvenlafaxine (NDV), N,O-didesmethylvenlafaxine (DDV), and N,N,O-tridesmethylvenlafaxine (TDV). In vitro studies indicate that the formation of ODV is catalyzed by CYP2D6; this has been confirmed in a clinical study showing that patients with low CYP2D6 levels (“poor metabolizers”) had increased levels of venlafaxine and reduced levels of ODV compared to people with normal levels of CYP2D6 (“extensive metabolizers”). The differences between CYP2D6 poor and extensive metabolizers, however, are not expected to be clinically important because the sum of venlafaxine and ODV is similar in the two groups and venlafaxine and ODV are pharmacologically approximately equiactive and equipotent. Approximately 87% of a venlafaxine dose is recovered in the urine within 48 hours as unchanged venlafaxine (5%), unconjugated ODV (29%), conjugated ODV (26%), or other minor active metabolites (27%). Renal elimination of venlafaxine and its metabolites is the primary route of excretion. The metabolic pathway of venlafaxine can be summarized as follows:

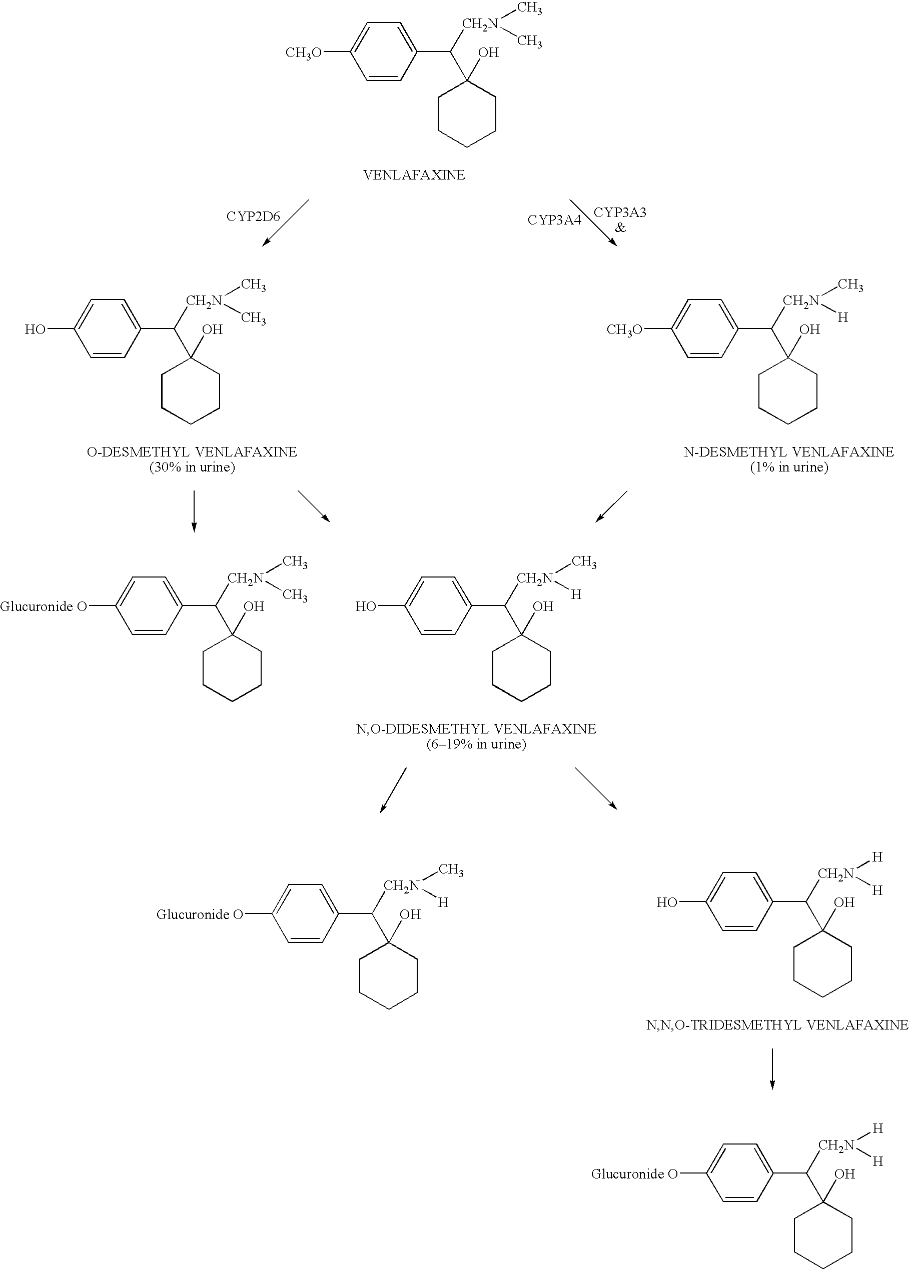

Venlafaxine's elimination half-life of about 4 hours is short, and its active metabolite has a half-life of about 8 hours. This results in venlafaxine being administered twice daily and a lack of patient compliance in keeping to this daily dosing schedule is liable to produce discontinuation problems. Sudden discontinuation of venlafaxine can result in withdrawal symptoms, which can include, fatigue, dizziness, nausea, headache and dysphoria. Accordingly, venlafaxine is an excellent candidate for a controlled-release oral formulation.

Venlafaxine, as its hydrochloride salt, is available as a second-generation extended-release tablet and is marketed under the brand name Effexor® XR for once daily use. Such a formulation has eliminated the discontinuation problems seen with Effexor®, the first-generation immediate-release form of venlafaxin, which is usually administered twice daily. Extended-release formulations of venlafaxine have been described in the prior art.

U.S. Pat. Nos. 6,274,171, 6,403,120, and 6,419,958, for example, disclose formulations comprising a therapeutically effective amount of venlafaxine hydrochloride in film-coated spheroids. The spheroids comprise a core having venlafaxine hydrochloride, microcrystalline cellulose, and optionally hydroxypropylmethylcellulose. The cores are coated with a mixture of ethylcellulose and hydroxypropylmethylcellulose and subsequently packaged into hard gelatin capsules. These patents also describe and claim methods and compositions for obtaining therapeutic blood plasma concentrations of venlafaxine over a twenty-four hour period with diminished incidence of nausea and emesis which comprise administering orally to a patient in need thereof, an extended-release formulation providing a peak blood plasma level of venlafaxine of no more than about 150 ng/ml 4-8 hours after administration.

U.S. Pat. No. 6,703,044 purports to teach a formulation wherein a delayed-burst release of venlafaxine is achieved at least three hours after administration resulting in dispersion of the venlafaxine mainly through the colon into the blood stream as a result of colon absorption over a period of at least 24 hours. A compressed core comprising a burst controlling agent as well as a disintegrant characterizes the formulation. The core is coated with a relatively rigid water insoluble, hydrophobic polymer, in which particles of water insoluble but hydrophilic material are embedded. These particles form channels upon contact with aqueous medium, which imbibe liquid and cause the burst-controlling agent to burst the coating thereby enabling the delayed-burst release of the venlafaxine. The '044 patent also teaches in Example 11 that the formulation surprisingly provided for a 30% higher bioavailability of the venlafaxine in fasting volunteers when compared to extended-release formulations of venlafaxine presently available on the market. The label for Effexor® XR, on the other hand, states that: “Effexor XR should be administered in a single dose with food either in the morning or evening at approximately the same time each day”. Example 11, the only pharmacokinetic study presented in the patent, does not show any bioavailability data in fed volunteers, and hence it is not known whether the formulation taught in the '044 patent will also provide for a higher bioavailability when administered to patients under the conditions recommended by the Effexor® XR label, i.e. under fed conditions. The '044 patent does not provide any data on the adverse events or side effect profile of the claimed composition.

The disclosures of the '120, '171, and '958 patents discussed above teach that “ . . . various attempts to produce extended release tablets of venlafaxine hydrochloride by hydrogel technology proved to be fruitless because the compressed tablets were either physically unstable (poor compressibility or capping problems) or dissolved too rapidly in dissolution studies.” Col. 4, lines 60-64 of the '120, 171, and '958 patents. Makhija and Vavia of the Pharmaceutical Division, Dept. of Chemical Technology (Autonomous), University of Mumbai, India, however, describe a once daily sustained-release tablet of venlafaxine using hydrogel technology (Eur. J. Pharmaceut. Biopharmaceut. 2002. 54:9-15). The Makhija and Vavia reference teaches a once daily sustained-release tablet of venlafaxine hydrochloride using an uncoated matrix system based on swellable as well as non-swellable polymers. Interestingly, the bioavailability of venlafaxine for this formulation, like that of the '044 formulation is, also significantly improved over that of Effexor® XR even though there does not appear to be any delay in the release of the drug in vitro (FIG. 2) or in vivo (FIG. 4). However, like the '044 invention, the formulation was administered to individuals in the fasted state. Accordingly, it is not known whether the Makhija and Vavia formulation would provide a higher bioavailability in the fed state. Finally, the Makhija and Vavia reference does not teach the effect of their formulation on the incidence and frequency of any adverse events in comparison to Effexor® XR.

Venlafaxine is currently among the top five prescribed antidepressant medications within the SSRI/SNRI category of antidepressants. However, only one once-a-day oral dosage form comprising venlafaxine hydrochloride is currently being marketed under the trade name Effexor® XR. Given the efficacy of venlafaxine, a once-a-day oral composition comprising at least one form of venlafaxine capable of providing a higher bioavailability compared to the currently marketed, Effexor® XR 150 mg capsules, with a reduced or similar side effect or adverse event profile would be desirable. Such a composition can also allow for a composition having an absolute amount of the active drug that is less that the amount in the reference product, thereby providing for an better safety profile.

### SUMMARY OF THE INVENTION

The present invention relates to a modified release composition of at least one form of venlafaxine.

In one embodiment of the invention, the modified release composition of the at least one form of venlafaxine is an enhanced absorption delayed controlled release pharmaceutical composition for oral administration suitable for once daily dosing comprising: a) a core comprising at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, and pharmaceutically acceptable excipient; and b) a modified release coating which substantially surrounds said core, wherein said composition provides enhanced absorption delayed controlled release of said at least one form of venlafaxine such that the combined geometric mean ratio of the composition of the invention to the reference product for the AUC₀₋ₜ or the Cₘₐₓ for venlafaxine or its active metabolite O-desmethylvenlafaxine is greater than 1 after first administration under fed or fasting conditions.

As used herein, the “geometric mean ratio” refers to the geometric mean of the composition of the invention divided by the geometric mean of the reference product for a particular pharmacokinetic parameter. Thus, the “geometric mean ratio” for the AUC₀₋ₜ for venlafaxine, for example, means the geometric mean of the AUC₀₋ₜ for venlafaxine of the composition of the invention divided by the geometric mean of the AUC₀₋ₜ for venlafaxine of the reference product. Thus, if the geometric mean for the AUC₀₋ₜ for venlafaxine of the composition of the invention is X and the geometric mean for the AUC₀₋ₜ for venlafaxine for reference product is Y, then the geometric mean ratio for the AUC₀₋ₜ for venlafaxine is X/Y. Similarly, if the geometric mean for the AUC₀₋ₜ for O-desmethylvenlafaxine of the composition of the invention is A and the geometric mean for the AUC₀₋ₜ for O-desmethylvenlafaxine of the reference product is B, then the geometric mean ratio for the AUC₀₋ₜ for O-desmethylvenlafaxine is A/B. As used herein, the “combined geometric mean ratio” means the geometric mean ratio of venlafaxine for a particular pharmacokinetic parameter plus the geometric mean ratio of O-desmethylvenlafaxine for the same pharmacokinetic parameter. To use the above example, the combined geometric mean ratio for the AUC₀₋ₜ is therefore [(X/Y)+(A/B)].

The term “first administration” as used herein means the first single dose of the composition of the invention administered to a patient or the first dose administered to a patient after a suitable washout period.

In another embodiment of the invention, the enhanced absorption delayed controlled release pharmaceutical composition for oral administration suitable for once daily dosing comprises: a) a core comprising at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, and pharmaceutically acceptable excipient; and b) a coating substantially surrounding said core, said coating comprising a water-insoluble water-permeable film-forming polymer, a water-soluble polymer or substance, and a plasticizer, wherein said composition provides enhanced absorption delayed controlled release of said at least one form of venlafaxine such that the combined geometric mean ratio of the composition of the invention to the reference product for the AUC₀₋ₜ or the Cₘₐₓ for venlafaxine or its active metabolite O-desmethylvenlafaxine is greater than 1 after first administration under fed or fasting conditions.

In another embodiment, the enhanced absorption delayed controlled release pharmaceutical composition for oral administration suitable for once daily dosing comprises: a) a core comprising at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, and pharmaceutically acceptable excipient; and; and b) a coating substantially surrounding said core, said coating comprising a water-insoluble water-permeable film-forming polymer, a water-soluble polymer or substance, and a plasticizer, wherein said composition provides an in vitro dissolution profile using the USP Type I apparatus method at 75 rpm in 1000 ml phosphate buffer pH 6.8 at 37° C. characterized by the equation:
y=100−100*e^{(-a*x}^{^{b}}⁾
where,
y=% dissolution,
x=sampling time,
a=scale parameter which ranges from about 0.07 to about 0.0004,
b=shape parameter which ranges from about 1.48 to about 3.02, and
100=the cumulative percentage of the at least one form of venlafaxine released at time infinity

In another embodiment of the invention, the enhanced absorption delayed controlled release pharmaceutical composition for oral administration suitable for once daily dosing comprises: a) a core comprising at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, and pharmaceutically acceptable excipient; and b) a coating substantially surrounding said core, said coating comprising a water-insoluble water-permeable film-forming polymer, a water-soluble polymer or substance, and a plasticizer, wherein said composition provides enhanced absorption delayed controlled release of said at least one form of venlafaxine such that the geometric mean ratio of the composition of the invention to the reference product for the AUC₀₋ₜ and/or the Cₘₐₓ for venlafaxine is greater than 2 after first administration under fed or fasting conditions.

In another embodiment of the invention, the enhanced absorption delayed controlled release pharmaceutical composition for oral administration suitable for once daily dosing comprises: a) a core comprising at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, and pharmaceutically acceptable excipient; and b) a coating substantially surrounding said core, said coating comprising a water-insoluble water-permeable film-forming polymer, a water-soluble polymer or substance, and a plasticizer, wherein said composition provides enhanced absorption delayed controlled release of said at least one form of venlafaxine such that the geometric mean ratio of the composition of the invention to the reference product for the AUC₀₋ₜ and/or the Cₘₐₓ for O-desmethylvenlafaxine is greater than 2 after first administration under fed or fasting conditions.

In one embodiment of the invention, the combined geometric mean ratio for the AUC₀₋ₜ is about 2.32 when the composition of the invention is administered under fed conditions.

In one embodiment of the invention, the combined geometric mean ratio for the AUC₀₋ₜ is about 2.33 when the composition of the invention is administered under fasting conditions.

In one embodiment of the invention, the combined geometric mean ratio for the Cₘₐₓ is about 2.65 when the composition is administered under fed conditions.

In one embodiment of the invention, the combined geometric mean ratio for the Cₘₐₓ is about 2.38 when the composition is administered under fasting conditions.

In one embodiment of the invention, the Tₘₐₓ of the composition of the invention compared to the reference product for venlafaxine is delayed by about 5 hours when the composition is administered under fed conditions.

In one embodiment of the invention, the Tₘₐₓ of the composition of the invention compared to the reference product for O-desmethylvenlafaxine is delayed by about 2 hours when the composition is administered under fed conditions.

In one embodiment of the invention, the Tₘₐₓ of the composition of the invention compared to the reference product for venlafaxine or O-desmethylvenlafaxine is delayed by about 2 hours under fasting conditions when the composition is administered under fasting conditions.

In one embodiment of the invention, the Tₘₐₓ for venlafaxine or O-desmethylvenlafaxine is greater than about 8 hours after first administration of the composition in the fed or fasted sate.

In one embodiment of the invention, the Tₘₐₓ for venlafaxine is at about 11 hours after first administration of the composition in the fed state.

In one embodiment of the invention, the Tₘₐₓ for O-desmethylvenlafaxine is at about 12 hours after first administration of the composition in the fed state.

In one embodiment of the invention, the Tₘₐₓ for venlafaxine is at about 10 hours after first administration of the composition in the fasted state.

In one embodiment of the invention, the Tₘₐₓ for O-desmethylvenlafaxine is at about 14 hours after first administration of the composition in the fasted state.

In one embodiment of the invention, the composition provides a Cₘₐₓ greater than 150 ng/ml for venlafaxine or O-desmethylvenlafaxine after first administration of the composition in the fed state.

In one embodiment of the invention, the composition provides a Cₘₐₓ of about 160 ng/ml for venlafaxine after first administration of the composition in the fed state.

In one embodiment of the invention, the composition provides a Cₘₐₓ of about 211 ng/ml for O-desmethylvenlafaxine after first administration of the composition in the fed state.

In one embodiment of the invention the pharmaceutically acceptable salt of venlafaxine is selected from the group consisting of venlafaxine hydrochloride, venlafaxine besylate, venlafaxin maleate, and venlafaxin fumarate.

In one embodiment of the invention, the pharmaceutically acceptable salt of venlafaxin is the hydrochloride salt of venlafaxine.

In one embodiment of the invention, the active metabolite of venlafaxine is O-desmethylvenlafaxine

In one embodiment t of the invention, the pharmaceutically acceptable salt of an active metabolite of venlafaxine is O-desmethylvenlafaxine succinate.

In one embodiment of the invention, the at least one gelling agent is selected from the group consisting of hydroxypropylmethylcellulose, hydroxypropylcellulose, polyethylene oxide, polyvinylpyrrolidone, carbomers, carragheen, polyvinylalcohol and mixtures thereof. It is preferable that the at least one gelling agent comprises by weight from about 10 to about 80%, preferably from about 10 to about 40% and most preferably about 21% by weight of the core dry weight. The at least one gelling agent preferably comprises a mixture of at least two gelling agents, most preferably hydroxypropylmethylcellulose (13%) and polyvinylpyrrolidone (8%).

In one embodiment of the invention, the core further comprises at least one filler selected from the group consisting of lactose monohydrate, anhydrous lactose, mannitol, sorbitol, microcrystalline cellulose, dibasic calcium, calcium sulfate and mixtures thereof. The at least one filler comprises up to about 75% by weight of the core dry weight. Preferably, the filler is lactose monohydrate, specifically Lactose # 315 Spray Dried, and comprises by weight about 23% by weight of the core dry weight.

In one embodiment of the invention, the core further comprises at least one lubricant selected from the group consisting of magnesium stearate, talc, stearic acid, sodium stearyl fumarate, calcium stearate, vegetable oil, silica gel, colloidal silicon dioxide, Compritol 888 ATO, and mixtures thereof. The at least one lubricant comprises from about 0.02 to about 5%, preferably from about 0.5 to about 2%, more preferably from about 0.5 to about 1% by weight of the core dry weight. The filler is most preferably magnesium stearate and comprises about 0.65% of the core dry weight.

In one embodiment of the invention, the modified release coating for the enhanced absorption delayed controlled release of the at least one form of venlafaxine provides an in vitro release profile, using the USP type I method at 75 rpm in 1000 ml phosphate buffer pH 6.8 at 37° C., characterized by the equation:
y=100−100*e^{(-a*x}^{^{b}}⁾
where,
y=% dissolution,
x=sampling time,
a=scale parameter which ranges from about 0.07 to about 0.0004,
b=shape parameter which ranges from about 1.48 to about 3.02, and
100=the cumulative percentage of the active released at time infinity.
   Preferably, the modified release coat comprises by weight based on the coating weight, about 20 to about 85% of at least one water-insoluble water-permeable film-forming polymer, about 10 to about 75% of at least one water-soluble polymer or substance, and about 3 to about 40% of at least one plasticizer. Preferably, the at least one form of venlafaxine is venlafaxine hydrochloride.

In one embodiment of the invention, the at least one water-insoluble water-permeable film-forming polymer is selected from the group consisting of ethylcellulose, cellulose acetate, methacrylic acid derivatives, Surelease®, Acryl-EZE®, and combination thereof. Preferably the at least one water-insoluble, water-permeable film-forming polymer comprises ethylcellulose by weight from about 55 to about 62% of the coating based on the coating weight. Most preferably, the ethylcellulose comprises by weight about 55% of the coating weight.

In one embodiment of the invention, the at least one water-soluble polymer is selected from the group consisting of polyvinylpyrrolidone, polyethyleneglycol, hydroxypropylmethylcellulose, hydrated colloidal silica, sucrose, mannitol, and combinations thereof. Preferably the at least one water-soluble polymer is polyvinylpyrrolidone and comprises by weight from about 26 to about 32% of the coating based on the coating weight. Most preferably, the polyvinylpyrrolidone comprises about 32% by weight of the coating weight.

In one embodiment of the invention, the at least one plasticizer is selected from the group consisting of citrate esters, dibutyl sebacate, dibutyl pthalate, triacetin, castor oil, polyalkyleneglycol, fatty acids, and combinations thereof. Preferably, the at least one plasticizer is stearic acid and comprises by weight from about 13 to about 14% of the coating weight. Most preferably, the stearic acid comprises about 13.5% by weight of the coating weight.

In one embodiment of the invention, the weight gain resulting from the application of the delayed and extended release coating onto the core ranges from about 2 to about 50%, preferably from about 2 to about 20%, more preferably from about 7.5 to about 10%, and most preferably about 8% of the core dry weight.

In one embodiment of the invention, the weight proportions of the water-insoluble water-permeable film forming polymer:water-soluble polymer:plasticizer is preferably about 50-85:10-40:5-20, more preferably about 55-62:26-32:13:14, and most preferably about 55:32:13.5.

In another embodiment of the invention, the oral dosage form when tested in vitro using the USP type I method at 75 rpm in 1000 ml phosphate buffer at pH 6.8 at 37° C., releases venlafaxine hydrochloride such that the release profile of the venlafaxine hydrochloride is characterized by the equation:
y=100−100*e^{(-a*x}^{^{b}}⁾
where,
y=% dissolution,
x=sampling time,
a=scale parameter which ranges from about 0.07 to about 0.0004,
b=shape parameter which ranges from about 1.48 to about 3.02, and
100=the cumulative percentage of venlafaxine hydrochloride released at time infinity.

In yet another embodiment of the invention, the oral dosage form, when tested in vitro using the USP type I method at 75 rpm in 1000 ml phosphate buffer at pH 6.8 at 37° C., provides a dissolution rate such that between about 0% and about 6.8% venlafaxine hydrochloride is released after about 1 hour, about 0.5% to about 18% is released after about 2 hours, about 3% to about 42% is released after about 4 hours, about 9% to about 63% is released after about 6 hours, about 19% to about 78% is released after about 8 hours, about 34% to about 88% is released after about 10 hours, about 52% to about 94% is released after about 12 hours, and no less than about 100% is released after about 18 hours.

In another embodiment of the invention, the oral dosage form when administered to a patient in need thereof provides a similar or diminished incidence of adverse events not influenced by food in comparison to the reference product.

### BRIEF DESCRIPTION OF THE DRAWINGS

The present invention will be further understood from the following detailed description with reference to the following drawings in which:

FIG. 1 is a graph illustrating the dissolution profile of uncoated cores according to an embodiment of the invention.

FIG. 2 is a comparative graph illustrating the dissolution profile of the core of FIG. 1 coated with three different coat compositions in comparison with the dissolution profile of the reference product.

FIG. 3 is a comparative graph illustrating the mean concentration-time profile of venlafaxine after single-dose administration of an oral dosage form of the invention comprising 150 mg venlafaxine hydrochloride in comparison with the reference product under fed conditions.

FIG. 4 is a comparative graph illustrating the mean concentration-time profile of O-desmethylvenlafaxine after single-dose administration of the oral dosage form of FIG. 3 in comparison with the reference product under fed conditions.

FIG. 5 is a comparative graph illustrating the mean concentration-time profile of N-desmethylvenlafaxine after single-dose administration of the oral dosage form of FIG. 3 in comparison with the reference product under fed conditions.

FIG. 6 is a comparative graph illustrating the mean concentration-time profile of N,O-didesmethylvenlafaxine after single-dose administration of the oral dosage form of FIG. 3 comprising 150 mg venlafaxine hydrochloride in comparison with the reference product under fed conditions.

FIG. 7 is a comparative graph illustrating the mean concentration-time profile of N,N,O-tri-desmethylvenlafaxine after single-dose administration of the oral dosage form in comparison with the reference product under fed conditions.

FIG. 8 is a comparative graph illustrating the mean concentration-time profile of venlafaxine after single-dose administration of an oral dosage form of the invention comprising 150 mg venlafaxine hydrochloride in comparison with the reference product under fasting conditions.

FIG. 9 is a comparative graph illustrating the mean concentration-time profile of O-desmethylvenlafaxine after single-dose administration of the oral dosage form of FIG. 8 in comparison with the reference product under fasting conditions.

FIG. 10 is a comparative graph illustrating the mean concentration-time profile of N-desmethylvenlafaxine after single-dose administration of the oral dosage form of FIG. 8 comprising 150 mg venlafaxine hydrochloride in comparison with the reference product under fasting conditions.

FIG. 11 is a comparative graph illustrating the mean concentration-time profile of N,O-didesmethylvenlafaxine after single-dose administration of the oral dosage form of FIG. 8 in comparison with the reference product under fasting conditions.

FIG. 12 is a comparative graph illustrating the mean concentration-time profile of N,N,O-tri-desmethylvenlafaxine after single-dose administration of the oral dosage form of FIG. 8 in comparison with the reference product under fasting conditions.

FIG. 13 is a comparative graph illustrating the dissolution profile of coated cores according to an embodiment of the invention in purified water.

FIG. 14 is a comparative graph illustrating the dissolution profile of coated cores of FIG. 13 in phosphate buffer pH 6.8 buffer.

### DETAILED DESCRIPTION OF THE INVENTION

The present invention is directed to a modified release pharmaceutical composition of venlafaxine. In particular, the composition is an enhanced absorption delayed controlled release composition of the at least one form of venlafaxine comprising a core and a modified release coating, which substantially surrounds the core, wherein the composition provides enhanced absorption delayed controlled release of the at least one form of venlafaxine.

The Tablet Cores

The core comprises at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, a gelling agent and optionally conventional excipients, surrounded by a polymer coat. The composition provides an enhanced absorption delayed controlled release of the at least one form of venlafaxine. The enhanced absorption delayed controlled release oral dosage form of the invention has a higher bioavailability with reduced or similar side effects or adverse events when compared to the reference product.

The proportion of the at least one form of venlafaxine in the core is present from about 10 to about 70%, preferably from about 25 to about 60%, and most preferably about 55% by weight of the core dry weight. The composition comprises a pharmaceutically effective amount of the at least one form of venlafaxin that can vary from about 0.5 to about 1000 mg, preferably from about 5 to about 500 mg, and most preferably from about 100 to about 200 mg.

The term “effective amount” as used herein means that a “pharmaceutically effective amount” is contemplated. A “pharmaceutically effective amount” is the amount or quantity of the at least one form of venlafaxine in a dosage form of the invention sufficient to elicit an appreciable clinical or therapeutic response when administered, in single or multiple doses, to a patient in need thereof. It will be appreciated that the precise therapeutic dose will depend on the age and condition of the patient and the nature of the condition to be treated and will be at the ultimate discretion of the attendant physician. It is well known to the skilled artisan that the therapeutically or clinically effective amount for a certain indication can be determined by conducting clinical studies using dosage forms that contain a pharmaceutically effective amount of the at least one form of venlafaxine.

As used herein, the term “pharmaceutically acceptable salts” refers to salts prepared from pharmaceutically acceptable non-toxic acids, including inorganic acids and organic acids. Suitable non-toxic acids include inorganic and organic acids such as acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesunfonic and the like. The hydrochloric salt is the most preferred. Other salts, such as venlafaxine maleate and venlafaxine besylate have been described in International patent application Nos. PCT/EP03/03319 (WO 03/082805) and PCT/EP03/03318 (WO 03/082804) respectively, the contents of which are incorporated herein by reference.

Venlafaxine, or the venlafaxine in the pharmaceutically acceptable salts of venlafaxine, can be any form of venlafaxine. For example, venlafaxine has one optically active carbon, thus allowing for existence of two enantiomers and a racemate. Both enantiomers are pharmaceutically active. Thus, the effective amount of the preferred active in the core of the oral dosage form of the invention, venlafaxine hydrochloride, can be based on the racemate or mixture of enantiomers of venlafaxine or on the pure or substantially pure (+) or (−) enantiomer of venlafaxine. The (+) and (−) enantiomers of venlafaxin have been described in U.S. Pat. Nos. 6,197,828 and 6,342,533 respectively the contents of which are incorporated herein by reference. All such forms of venlafaxine are included within the meaning of the term “venlafaxine”, “pharmaceutically acceptable salts of venlafaxine”, “active metabolite of venlafaxine”, and “pharmaceutically acceptable salts of an active metabolite of venlafaxine”.

The at least one gelling agent comprises a substance that is hydrophilic in nature and which is capable of behaving like a hydrophilic matrix. Non-limiting examples of gelling agents include, but are not limited to, hydroxypropylmethylcellulose, hydroxypropylcellulose, polyethylene oxide, polyvinylpyrrolidone, xanthan gum, carbomers, carragheen, and polyvinyl alcohol. The at least one gelling agent can vary from about 10 and about 80%, preferably from about 10 and about 40%, and most preferably about 21% by weight of the core dry weight. Preferably, the at least one gelling agent comprises a mixture of at least two gelling agents. Most preferably, the at least two gelling agents is a mixture of hydroxypropylmethylcellulose present at about 13% by weight of the core dry weight and polyvinylpyrrolidone present at about 8% by weight of the core dry weight.

In addition to the above ingredients, a series of excipients can be included in the tablet to ensure that the tabletting operation can run satisfactorily and to ensure that tablets of specified quality are prepared. Depending on the intended main function, excipients to be used in tablets are subcategorized into different groups. However, one excipient can affect the properties of a tablet in a series of ways, and many excipients used in tablet compositions can thus be described as being multifunctional.

For example, the core can further comprise at least one lubricant. Lubricants are added to pharmaceutical formulations to ensure that tablet formation and ejection can occur with low friction between the solid and the die wall. High friction during tabletting can cause a series of problems, including inadequate tablet quality (capping or even fragmentation of tablets during ejection, and vertical scratches on tablet edges) and can even stop production. Non-limiting examples of lubricants useful for the oral dosage form described herein include magnesium stearate, talc, sodium stearyl fumarate, calcium stearate, silica gel, colloidal silicon dioxide, Compritol 888 ATO, glyceryl behenate, stearic acid, hydrogenated vegetable oils (such as hydrogenated cottonseed oil (Sterotex®), hydrogenated soybean oil (Sterotex® HM) and hydrogenated soybean oil & castor wax (Sterotex® K), stearyl alcohol, leucine, polyethylene glycol (MW 4000 and higher), and mixtures thereof. The at least one lubricant can be present in an amount from about 0.02 to about 5% by weight of the core dry weight. The preferred lubricant is magnesium stearate and is preferably present at about 0.65% by weight of the core dry weight.

Some oral dosage forms require the incorporation of one or more excipients into the dosage form to increase the bulk volume of the powder and hence the size of the dosage form. Accordingly, the core can further comprise at least one filler (or diluent). Non-limiting examples of the at least one filler useful for the oral dosage form described herein include lactose monohydrate, anhydrous lactose, mannitol, sorbitol, microcrystalline cellulose, dibasic calcium, and calcium sulfate. Mixtures of fillers can also be used. The at least one filler is preferably present up to about 75% by weight of the core dry weight. The preferred filler is lactose monohydrate. Most preferably, the lactose monohydrate is of the type called Lactose #315 Spray Dried, which is a mixture of a specially prepared pure α-lactose monohydrate along with a small amount of amorphous lactose. Preferably, the Lactose #315 Spray Dried is present at about 23% by weight of the core dry weight.

The at least one form of venlafaxine, and optionally, the filler are first dry blended in a high shear mixer such as a Fielder PMA 65. The dry blend is then granulated using a wet granulation process. The preferred granulating aid or binder used is a solution of polyvinylpyrrolidone dissolved in isopropyl alcohol 99% USP, which is sprayed onto the dry blend. The polyvinylpyrrolidone, which as described above is a preferred gelling agent, also functions as a granulating aid. The wet granules formed are dried overnight (about 16 hours) at about 45±5° C. and subsequently milled in a Comil fitted with a 0.062-inch screen. The sieved granules are then blended with the lubricant, preferably magnesium stearate, and if necessary, any other additional inert excipients, which can improve processing of the oral dosage form of the invention. Blending of the granules with the lubricant, and if necessary, any additional inert excipients, such as for example a glidant, may be performed in a V-blender or any other suitable blending apparatus.

The dried milled granules are then pressed into tablets and are hereinafter referred to as “tablet cores” or simply as “cores”. The tablet cores have a hardness ranging from about 7 to about 15 KP. Tablet cores can be obtained by the use of standard techniques and equipment well known to the skilled artisan. Preferably, the tablet cores are obtained by a rotary press (also referred to as a multi-station press) fitted with suitable punches. At this stage, the core formulation is an imrnediate-release formulation resulting in greater than about 90% release of the at least one form of venlafaxine in about 30 minutes.

The cores are next coated with a polymer coat for the enhanced absorption delayed controlled-release of the at least one form of venlafaxine. The coat is designed to achieve an in vitro release profile of the at least one form of venlafaxine, preferably the hydrochloride salt of venlafaxine, such that the composition, when tested in vitro using the USP type I method at 75 rpm in 1000 ml phosphate buffer pH 6.8 at 37° C., provides a release profile characterized by the following equation:
y=100−100*e^{(-a*x}^{^{b}}⁾
where,
y=% dissolution,
x=sampling time,
a=scale parameter which ranges from about 0.07 to about 0.0004,
b=shape parameter which ranges from about 1.48 to about 3.02, and
100=the cumulative percentage of venlafaxine hydrochloride released at time infinity.
   The mathematical function y=100−100*e^{(-a*x}^{^{b}}⁾ is well known in the art as a Weibull distribution (Polli J et al. Drug Info. J 30:1113-1120, 1996; Costa, P. and Lobo, J. M. S. E. J. Pharm. Sci. 123-133, 2001; Lagenbucher, F. J. Pharm. Pharmac. 24:979, 1972). Without wishing to be bound by theory, it is believed that the release profile, obtained by the polymer coat designed to obtain a dissolution profile characterized by the above equation, may be responsible for the similar or diminished incidence of adverse events not influenced by food in comparison to the reference product, Effexor® XR, even though the composition of the invention provides for a higher bioavailability of the active compared to Effexor® XR. The polymer coat is also designed such that the integrity of the coat remains intact and does not dissolve and/or disintegrate for a period of at least about 24 hours in purified water, 0.1 N HCl, Simulated Gastric Fluid (SGF) pH 1.2, or pH 6.8 phosphate buffer. As these conditions are intended to mimic the in vivo condition, it is believed that the integrity of the polymer coat will also remain intact and not dissolve and/or disintegrate in the gastrointestinal tract. The polymer coat is thus fundamentally different from the polymer coat described in U.S. Pat. No. 6,117,453, which is a quick-dissolving film, and U.S. Pat. No. 6,703,044, which is a rigid film designed to burst, thereby releasing the active from the core. In summary, the polymer coat for achieving the enhanced absorption delayed controlled release of the at least one form of venlafaxine is designed to provide a release profile characterized by the Weibull distribution shown above and is not a quick dissolving and/or disintegrating coat.

The preferred polymer coat for achieving the enhanced absorption delayed controlled-release of the at least one form of venlafaxine is a semi-permeable coat comprising at least one water-insoluble, water-permeable film-forming polymer, at least one water-soluble polymer or substance, and at least one plasticizer designed to achieve an in vitro release profile characterized by the Weibull distribution as defined above and does not dissolve and/or disintegrate for at least about a 24-hour period.

Non-limiting examples of the at least one water-insoluble, water permeable film-forming polymer can be a cellulose ether, such as ethylcellulose, a cellulose ester, such as cellulose acetate, methacrylic acid derivatives, aqueous ethylcellulose dispersions such as Surelease®, aqueous enteric coating systems such as Sureteric®, and aqueous acrylic enteric systems such as Acryl-EZE®. Combinations are also permitted. The at least one water-insoluble, water-permeable film forming polymer is present in an amount ranging from about 20 to about 85%, preferably from about 55 to about 62%, and most preferably about 55% by weight of the coating dry weight. Most preferably, ethylcellulose is the at least one water-insoluble, water-permeable film-forming polymer and is preferably present from about 55 to about 62% and most preferably at about 55% of the coating dry weight.

The at least one water-soluble polymer or substance can be a partially or totally water-soluble hydrophilic substance intended to modulate the film permeability to the outside aqueous medium. Non-limiting examples of the at least one water-soluble polymer or substance can be polyvinylpyrrolidone, polyethyleneglycol, hydroxypropylmethylcellulose, hydrated colloidal silica, sucrose, mannitol, and combinations thereof. The at least one water-soluble polymer comprises from about 10 to about 75%, preferably from about 26 to about 32% and most preferably about 32% by weight of the coating dry weight. Most preferably, the at least one water-soluble polymer is polyvinylpyrrolidone and comprises preferably from about 26 to about 32%, and most preferably at about 32% by weight of the coating dry weight.

Plasticizers are generally added to film coating formulations to modify the physical properties of the polymer to make it more usable. The amount and choice of the plasticizer contributes to the hardness of a tablet and may even affect its dissolution or disintegration characteristics, as well as its physical and chemical stability. One important property of plasticizers is their ability to make a coat elastic and pliable, thereby decreasing the coat's brittleness. Non-limiting examples of the at least one plasticizer useful for the preferred polymer coat include polyols, such as polyethylene glycol of various molecular weights, organic esters, such as diethyl phthalate or triethyl citrate, dibutyl sebacate, dibutyl pthalate, and oils/glycerides such as fractionated coconut oil or castor oil. Combinations are permitted. The at least one plasticizer is present from about 3 to about 40%, preferably from about 13 to about 14%, and most preferably about 13.5% by weight of the coating dry weight. The preferred at least one plasticizer is a fatty acid, specifically stearic acid, and is preferably present in an amount from about 13 to about 14%, and most preferably at about 13.5% by weight of the coating dry weight.

The relative proportions of the preferred polymer coat ingredients, notably the ratio of the at least one water-insoluble, water-permeable film-forming polymer:the at least one water-soluble polymer or substance:the at least one plasticizer, can be varied depending on the desired rate of release. The skilled artisan will appreciate that controlling the permeability and/or the amount of coating applied to the tablet cores can control the release of the active. For example, the permeability of the preferred polymer coat, can be altered by varying the ratio of the at least one water-insoluble, water-permeable film-forming polymer:the at least one water-soluble polymer:the at least one plasticizer and/or the quantity of coating applied to the tablet cores. A more delayed controlled-release is generally obtained with a higher amount of water-insoluble, water-permeable film forming polymer, a lower amount the at least one water soluble polymer, and/or by increasing the amount of the coating solution applied to the tablet cores. Alternatively, a faster rate of release can be obtained by increasing the amount of the water-soluble polymer, decreasing the amount of the at least one water-insoluble water permeable film-forming polymer, and/or by decreasing the amount of coating solution applied. The addition of other excipients to the tablet core can also alter the permeability of the coat. For example, if it is desired that the tablet core further comprise an expanding agent, the amount of plasticizer in the coat can be increased to make the coat more pliable as the pressure exerted on a less pliable coat by the expanding agent can rupture the coat. Other excipients such as pigments and taste-masking agents can also be added to the coating formulation. The preferred proportions of the at least one water-insoluble water-permeable film forming polymer:the at least one water-soluble polymer:the at least one plasticizer for maintaining the integrity of the coat for at least about 24 hours and for obtaining the release profile characterized by the Weibull equation described above is about 50-85:10-40:5-20.

The polymer coat was prepared and applied as follows. The appropriate amounts of the water-insoluble water-permeable film-forming polymer, preferably ethylcellulose, the water-soluble polymer, preferably, polyvinylpyrrolidone, and plasticizer, preferably stearic acid were all dissolved in an alcoholic solvent such as ethanol, isopropyl alcohol, or a mixture thereof. The resulting coating solution was sprayed onto the tablet cores, using a coating pan apparatus. The percentage weight gain resulting from application of the coating solution onto the cores can range from about 2 to about 50%, preferably from about 8 to about 30%, more preferably from about 10 to about 18% and most preferably about 15% by weight of the uncoated cores. Surprisingly, it was discovered that the above coating formulation provides for an enhanced absorption delayed controlled-release composition even though no pore-forming agent is present in the coating.

The following examples illustrate the present invention and are not intended to limit the scope of the present invention.

### EXAMPLE 1

Tablet Cores

The core formulation was made as shown in Table 1:

TABLE 1

| Ingredient | Mg/tablet | % w/w |
|---|---|---|
| Venlafaxine HCl | 169.71 | 55.10 |
| Filler¹ | 71.29 | 23.15 |
| Gelling agent² | 40.00 | 12.99 |
| Binder³ | 25.00 | 8.11 |
| Lubricant⁴ | 2.00 | 0.65 |
| Solvent⁵ | 85.00 | — |
| Total | 308.00 | 100.00 |

¹Lactose #315 Spray Dried
²Hydroxypropylmethylcellulose
³Polyvinylpyrrolidone
⁴Magnesium stearate
⁵Isopropyl alcohol 99% USP. Evaporates after drying

The venlafaxine hydrochloride, filler (Lactose #315 Spray Dried) and gelling agent (hydroxypropylmethylcellulose) were placed in a high shear mixer (Fielder PMA 65) and mixed at an impeller speed of about 200 rpm with the chopper speed at “I” for about 2 minutes. The impeller speed was then increased to 400 rpm with the chopper speed at “II” for an additional about 3 minutes. This mixture was then granulated with a solution of binder (polyvinylpyrrolidone) in isopropyl alcohol. The granules thus formed were then dried for about 16 hours at 45±5° C. The dried granules were next screened using a Comil fitted with a 0.062 inch screen. The screened granules were blended with the lubricant (magnesium stearate) for about 10 minutes in a V-blender and then compressed into tablets using a conventional rotary tablet press. The resulting tablets have a hardness ranging from about 7 to about 15 KP.

The dissolution of the resulting tablet cores was determined under the following dissolution conditions:
Medium: 1000 ml purified water
Method: USP Type I apparatus, 75 rpm at 37° C.

The results shown in Table 2 are presented as % released of the total venlafaxine hydrochloride in the tablet cores:

TABLE 2

| Time | % released | Std Dev | % RSD | Min | Max |
|---|---|---|---|---|---|
| 0 | 0 | 0 | 0 | 0 | 0 |
| 5 | 35 | 1 | 3 | 34 | 36 |
| 15 | 85 | 1 | 1 | 84 | 86 |
| 30 | 104 | 1 | 1 | 103 | 105 |
| 45 | 104 | 0 | 0 | 104 | 105 |
| 60 | 105 | 1 | 1 | 104 | 105 |

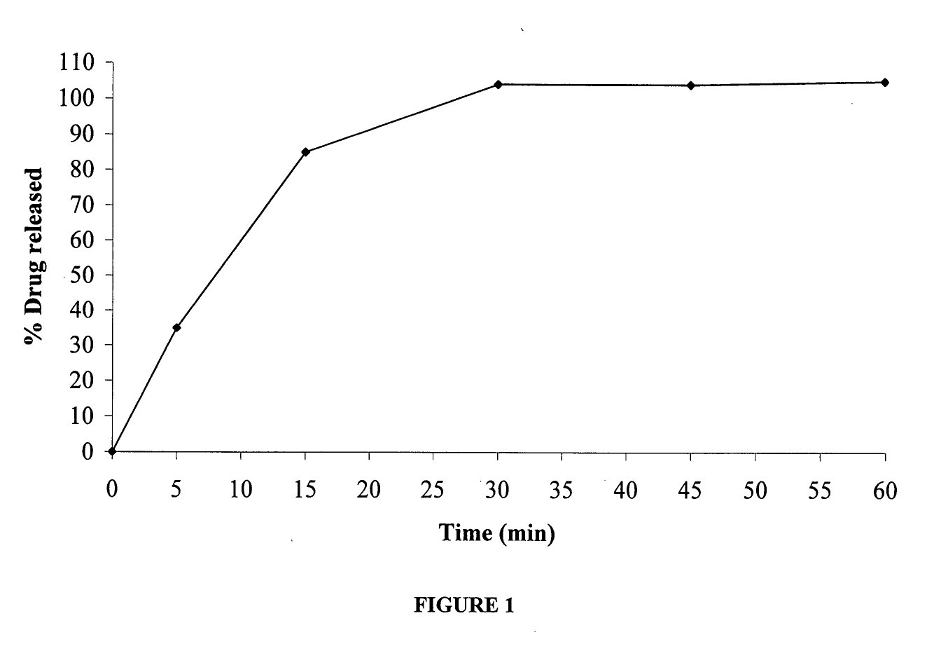

The data is further graphically presented in FIG. 1, which shows greater than 90% of the venlafaxine hydrochloride is released in about 30 minutes.

### EXAMPLE 2

Coating Formulation

Four coat formulations were made as shown in Table 3:

TABLE 3

| | Mg/tablet | | | |
|---|---|---|---|---|
| Ingredient | A | B | C | D |
| Water-insoluble water- | 12.650 | 13.750 | 16.500 | 15.217 |
| permeable film forming | | | | |
| polymer¹ | | | | |
| Water-soluble polymer² | 7.245 | 7.875 | 9.450 | 6.525 |
| Plasticizer³ | 3.105 | 3.375 | 4.050 | 3.258 |
| Solvent⁴ | 232.5604 | 252.783 | 303.340 | 252.783 |
| Total | 255.5604 | 277.783 | 333.340 | 277.783 |
| Dry solids | 23.000 | 25.000 | 30.000 | 25.000 |
| (% weight gain) | (7.5%) | (8.11%) | (9.74%) | (8.11%) |
| Tablet Cores (from Example 1) | 308.000 | 308.000 | 308.000 | 308.000 |
| (mg) | | | | |
| Total weight of coated tablet | 331.000 | 333.000 | 338.000 | 333.000 |

¹Ethocel 100 STD Premium
²Kollidon 90F
³Stearic Acid
⁴Ethyl alcohol 190 proof. Evaporates after drying, not included in total weight of coated tablets.

The plasticizer (stearic acid) was first dissolved in the solvent (ethyl alcohol). The water-insoluble water-permeable film-forming polymer (Ethocel 100 STD Premium) was slowly added to the plasticizer/ethanol mixture followed by the addition of the water-soluble polymer (Kollidon 90F) until a homogenous solution was achieved. Coating of the tablet cores from Example 1 was then carried out in an O'Hara Labcoat III system with the parameters shown in Table 4:

TABLE 4

| | | |
|---|---|---|
| Inlet Temperature (° C.) (for coating) | SV: | 40 ± 5 |
| | PV: | 40 ± 5 |
| Inlet Temperature (° C.) (for drying) | | 40 ± 5 |
| Exhaust Temperature (° C.) | | 35 ± 5 |
| Product Temperature (° C.) | | 35 ± 2 |
| ΔP Differential Pressure (IN. W.C.) | | −0.1 to −0.12 |
| Supply Air Flow (CFM) | | 200 ± 50 |
| Pan Speed (rpm) | | 2.5-15 |
| Atomizing Air (psi) | | 25-35 |
| Pattern Air (psi) | | 20-30 |
| Spraying Rate (g/min) | | 5-15 |

The tablets are coated until the desired weight gain was reached and subsequently dried at an exhaust temperature of exhaust temperature of 43±2° C., for 5 minutes at pan speed 3 rpm. Drying was continued for another 20 minutes at Jog with the same pan speed and the same parameters. The inlet temperature was subsequently turned off and the tablets cooled by keeping the exhaust on. The dissolution of the coated tablets, also referred to herein as “venlafaxine XR tablets, 150 mg”, “venlafaxine hydrochloride XR tablets, 150 mg”, the “test formulation” or as the “enhanced absorption delayed controlled release composition” was determined under the same experimental conditions as for the uncoated tablet cores compared to the reference product Effexor® XR. The results are presented in Table 5 as % released of the total venlafaxine hydrochloride in the coated tablet cores:

TABLE 5

| | Tablet | | | | Tablet | | | | Tablet | | | | |
|---|---|---|---|---|---|---|---|---|---|---|---|---|---|
| | cores | | | | cores | | | | cores | | | | |
| | coated with | | | | coated with | | | | coated with | | | | Effexor |
| Time | coat | | | | coat | | | Std | coat | | | Std. | 150 mg |
| (hrs) | formulation B | Min | Max | Std Dev | formulation C | Min | Max | Dev | formulation D | Min | Max | Dev | (n = 12) |
| 0 | 0 | 0 | 0 | 0 | 0 | 0 | 0 | 0 | 0 | 0 | 0 | 0 | 0 |
| 1 | 12 | 11 | 14 | 1 | 7 | 7 | 8 | 1 | 11 | 9 | 13 | 1 | 17 |
| 2 | 29 | 26 | 30 | 2 | 22 | 21 | 24 | 1 | 20 | 16 | 23 | 2 | 33 |
| 3 | 50 | 46 | 52 | 3 | 40 | 38 | 43 | 2 | 30 | 25 | 35 | 3 | 46 |
| 4 | 70 | 65 | 72 | 3 | 59 | 56 | 63 | 3 | 42 | 35 | 55 | 5 | 56 |
| 5 | 84 | 79 | 86 | 3 | 75 | 71 | 79 | 3 | 56 | 46 | 72 | 7 | 63 |
| 6 | 92 | 88 | 94 | 2 | 86 | 83 | 89 | 2 | 70 | 57 | 83 | 8 | 68 |
| 7 | 98 | 94 | 99 | 2 | 93 | 90 | 96 | 2 | 81 | 67 | 90 | 7 | 73 |
| 8 | 100 | 98 | 102 | 1 | 97 | 95 | 99 | 2 | 88 | 76 | 95 | 5 | 76 |
| 9 | 102 | 99 | 103 | 2 | 100 | 98 | 102 | 2 | 93 | 83 | 98 | 4 | 79 |
| 10 | 103 | 101 | 104 | 1 | 102 | 100 | 103 | 1 | 97 | 89 | 101 | 3 | 82 |
| 11 | 103 | 101 | 105 | 1 | 103 | 100 | 104 | 2 | 99 | 93 | 102 | 3 | 84 |
| 12 | 103 | 101 | 105 | 1 | 103 | 101 | 104 | 1 | 101 | 96 | 103 | 2 | 85 |
| 13 | 104 | 102 | 105 | 1 | 103 | 101 | 105 | 1 | 101 | 98 | 103 | 1 | 87 |
| 14 | 104 | 102 | 105 | 1 | 104 | 102 | 105 | 1 | 102 | 99 | 104 | 1 | 88 |
| 15 | 104 | 102 | 105 | 1 | 104 | 102 | 105 | 1 | 102 | 100 | 104 | 1 | 89 |
| 16 | 104 | 102 | 106 | 1 | 104 | 102 | 105 | 1 | 102 | 101 | 104 | 1 | 90 |
| 17 | 104 | 102 | 106 | 1 | 104 | 102 | 105 | 1 | 103 | 101 | 104 | 1 | 91 |
| 18 | 104 | 102 | 106 | 1 | 104 | 102 | 105 | 1 | 103 | 101 | 104 | 1 | 92 |

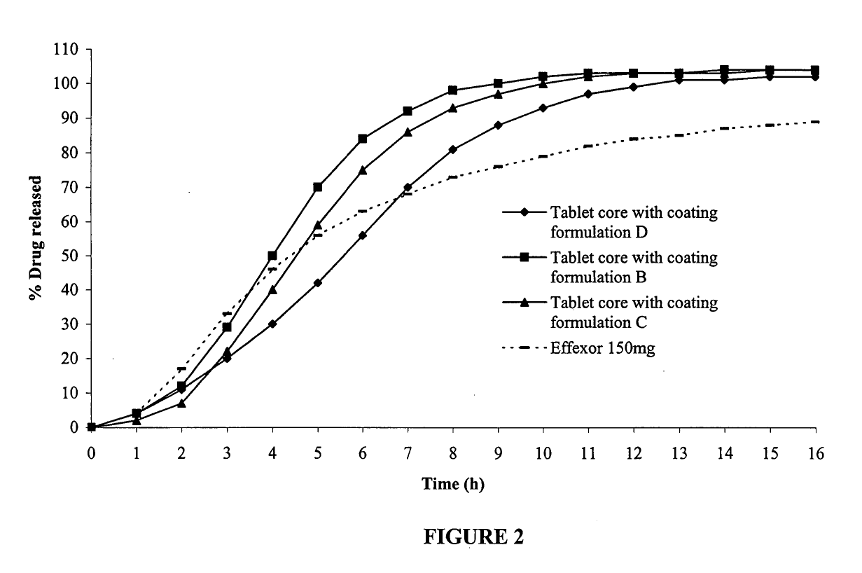

The data is further graphically presented in FIG. 2. The release profile of the coated tablet cores compared to the release profile of the uncoated cores shows that the polymers used in the granulation process to form the cores do not significantly impede the release of drug from the tablet. The polymer coat provides the enhanced absorption delayed controlled release profile.

### EXAMPLE 3

Pharmacokinetic Studies

### EXAMPLE 3A

The objective of this study was to compare the peak and systemic exposure of venlafaxine and its metabolites from a test formulation of venlafaxine hydrochloride 150 mg tablets of the invention versus the reference Effexor® XR 150 mg capsules under fed conditions. Bioavailability of these formulations was assessed for ODV, NDV, DDV, and TDV. In addition, urinary recovery of venlafaxine, ODV, NDV, DDV, TDV, ODV glucuronide and DDV glucuronide was compared between the composition of the invention and the reference product Effexor® XR 150 mg capsules.

The study design involved a two-way, crossover, open-label single-dose, fed, bioavailability study of 150 mg venlafaxine hydrochloride tablets of the invention versus the reference-product Effexor® XR 150 mg capsules in normal healthy non-smoking male subjects. Sixteen normal, healthy, non-smoking male subjects, within an age range of 18 to 65 years were selected the study after meeting several inclusion and exclusion criteria no more than 30 days prior to first administration of the composition of the invention and the reference product. All subjects underwent a medical history, medication history, physical examination (including blood pressure, heart rate and temperature) and ECG prior to starting the study. Selected routine clinical laboratory measurements, including screens for hepatitis C, hepatitis B-surface antigen, HIV, urine drugs of abuse, urine nicotine (cotinine) and saliva alcohol were performed during the screening. At check-in for each study period, screens for urine drugs of abuse, cotinine and saliva alcohol were performed on all subjects.

Subjects received one of the following treatments at 0.0 hour on Day 1 of each study period within 5 minutes of consuming a high-fat breakfast according to a computer generated randomization scheme:

Treatment A

Following an overnight fast of at least ten hours, one test formulation of venlafaxine hydrochloride XR 150 mg tablet of the invention was administered with 240 ml of ambient temperature water, 30 minutes after the start of the breakfast (Treatment Dose=150 mg).

Treatment B

Following an overnight fast of at least about 10 hours, one Effexor® XR 150 mg capsule was administered with 240 ml ambient temperature water, 30 minutes after the start of the breakfast (Treatment Dose=150 mg).

The study consisted of two 5-day study periods separated by a two-week washout period between treatments.

Water was provided ad libitum until 1.0 hour pre-dose. Fluid intake was controlled and consistent for 2.0 hours following drug administration as follows: drug was given with 240 ml of ambient temperature water. 150 ml of ambient temperature water was administered according to the following schedule: 2.0. 3.0, 4.0, 5.0, 6.0, 7.0, 8.0, 9.0, 10.0, 11.0, 12.0, 16.0, 20.0, 24.0, 28.0, 32.0, 36.0, 40.0, 44.0, 48.0, 52.0, 56.0, 60.0, 64.0 and 68.0 hours post-dose. No additional water was permitted outside of the scheduled time points, except for the 240 ml of water used to administer the composition of the invention and the additional fluid provided with meals. Each serving of water must be consumed within 5 minutes. In instances where time to void and time of water administration coincide, the subject was asked to void prior to consuming water.

Twenty blood samples (5 ml each) were drawn in each period for each treatment according to the following schedule: 0.0 (pre-dose), 1.0, 2.0, 3.0, 4.0, 5.0, 6.0, 7.0, 8.0, 9.0, 10.0, 12.0, 14.0, 16.0, 18.0, 24.0, 36.0, 48.0, 60.0 and 72.0 hours post-drug administration.

Urine was collected from all subjects during the following time intervals: Prior to dosing (complete void and collect), 0.0-2.0, 2.0-4.0, 4.0-8.0, 8.0-12.0, 12.0-24.0, 24.0-48.0 and 48.0-72.0 hours post-dose. For each individual subject, all urine samples for each specified time interval were collected and pooled. A minimum of 10 ml of urine was required for each time interval for each subject. For each time interval, the pH and volume of the pooled urine sample was measured and recorded.

The following pharmacokinetic parameters for venlafaxine, ODV, NDV, DDV, and TDV were calculated by standard non-compartmental methods: AUC₀₋ₜ, Cₘₐₓ, Tₘₐₓ, Clᵣ, Aₑ, and AUCₘₑₜ/AUCₚₐᵣₑₙₜ.

Descriptive statistics were performed for plasma and urine concentrations and for all PK parameters. Using GLM procedures in SAS, analysis of variance (ANOVA) was performed on log-transformed AUC₀₋ₜ, and Cₘₐₓ and on untransformed Tₘₐₓ, Clᵣ, Aₑ, AUCₘₑₜ/AUCₚₐᵣₑₙₜ at a significance level of 0.05. The intra-subject coefficient of variation (CV) was calculated using the Mean Square Error (MSE) from the ANOVA table. The ratio of geometric means and the 90% geometric confidence interval (90% C.I.) were calculated based on the difference in the Least Squares Means of the log-transformed AUC₀₋ₜ, and Cₘₐₓ between the test and reference formulations.

The term “Cₘₐₓ” as used herein is defined as the peak mean blood plasma concentration of the venlafaxine or an active metabolite of venlafaxine exhibited by the composition of the invention described herein. The mean Cₘₐₓ is calculated by adding the peak blood plasma concentration of venlafaxine or a metabolite of venlafaxine, of each of the subjects of a pharmacokinetic study divided by the number of subjects in the study.

The term “Tₘₐₓ” refers to the mean time to peak mean blood plasma concentration (Cₘₐₓ) of venlafaxine or a metabolite of venlafaxine.

The “AUC”, or the Area Under the Curve, of a pharmacokinetic profile, signifies the extent of absorption of a drug. The term “AUC₀₋ₜ” as used herein is the area under the blood plasma concentration-time curve from time 0 to time t for either venlafaxine or a metabolite of venlafaxine, where t is the last time point with measurable concentration for an individual formulation. Specifically, the term “AUCₘₑₜ” is the area under the blood-plasma concentration-time curve for a metabolite of venlafaxine and the term “AUCₚₐᵣₑₙₜ” is the area under the blood-plasma concentration-time curve for venlafaxine.

“Aₑ” or “Cumulative amounts excreted unchanged into urine” means the cumulative amount of venlafaxine or metabolite of venlafaxine excreted unchanged into the urine.

The incidence of all adverse events were tabulated by treatment group and subject number. MedDRA Version 6.1 was used to document all adverse events.

Tables 6-12 summarize data obtained for this study:

TABLE 6
(VENLAFAXINE)
I. Summary of pharmacokinetic results for venlafaxine in plasma and urine

| Pharmacokinetic | Treatment A | Treatment B |
|---|---|---|
| Parameters | Mean ± SD | Mean ± SD |
| AUC₀₋ₜ (ng · hr/mL) | 2081.31 ± 1463.31 | 1863.15 ± 1273.21 |
| Cₘₐₓ (ng/mL) | 162.08 ± 128.33 | 109.53 ± 56.53 |
| Tₘₐₓ (hr) | 10.07 ± 1.79 | 6.40 ± 2.32 |
| | (10.00*) | (5.00*) |
| Aₑ (mg) | 11.11 ± 7.72 | 9.92 ± 7.84 |
| CLᵣ (L/hr) | 5.65 ± 1.93 | 5.66 ± 1.90 |

II. Summary of bioavailability assessments for venlafaxine in plasma
(Treatment A v. Treatment B)

| | | Geometric Mean |
|---|---|---|
| | 90% Confidence Interval | Ratio |
| AUC₀₋ₜ | 103-126 | 1.14 |
| Cₘₐₓ | 106-154 | 1.29 |

*Median Value

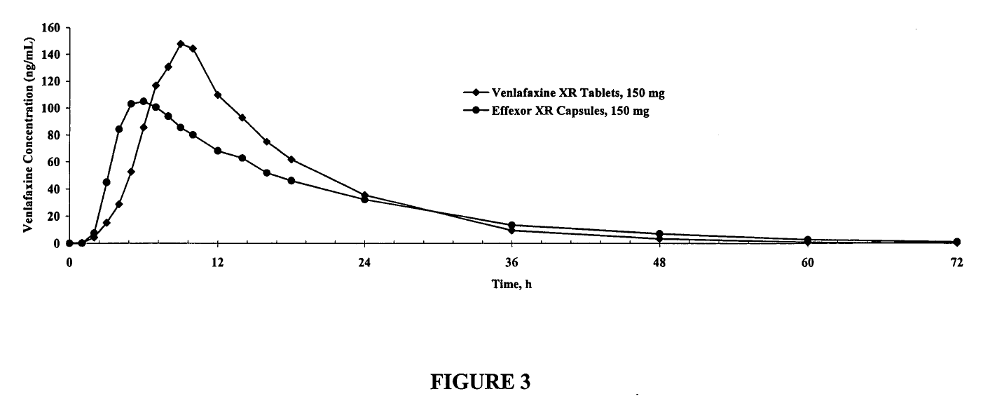

The blood-plasma concentration time curve for the above data is shown in FIG. 3.

TABLE 7
(O-DESMETHYLVENLAFAXINE)
I. Summary of pharmacokinetic results for O-desmethylvenlafaxine
in plasma and urine

| Pharmacokinetic | Treatment A | Treatment B |
|---|---|---|
| Parameters | Mean ± SD | Mean ± SD |
| AUC₀₋ₜ (ng · hr/mL) | 5283.33 ± 1800.90 | 4432.66 ± 1357.14 |
| Cₘₐₓ (ng/mL) | 211.46 ± 75.89 | 156.28 ± 56.37 |
| Tₘₐₓ (hr) | 12.53 ± 3.04 | 11.00 ± 2.33 |
| | (12.00*) | (10.00*) |
| Aₑ (mg) | 34.42 ± 10.93 | 30.70 ± 12.05 |
| CLᵣ (L/hr) | 6.85 ± 1.99 | 7.20 ± 2.55 |

II. Summary of bioavailability assessments for O-desmethylvenlafaxine
in plasma (Treatment A v. Treatment B)

| | | Geometric Mean |
|---|---|---|
| | 90% Confidence Interval | Ratio |
| AUC₀₋ₜ | 112-125 | 1.18 |
| Cₘₐₓ | 124-148 | 1.35 |

*Median Value

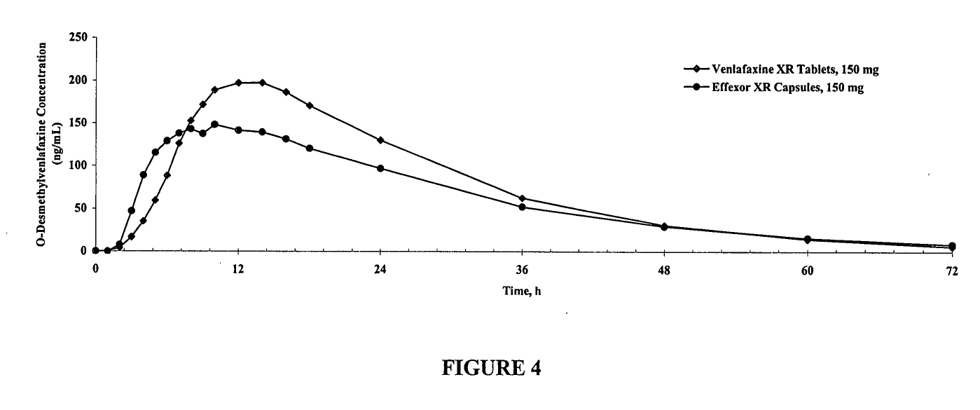

The blood-plasma concentration time curve for ODV from the composition of the invention versus that from Effexor® XR is shown in FIG. 4.

TABLE 8
(N,O-DIDESMETHYLVENLAFAXINE)
I. Summary of pharmacokinetic results for
N,O-didesmethylvenlafaxine in plasma and urine

| Pharmacokinetic | Treatment A | Treatment B |
|---|---|---|
| Parameters | Mean ± SD | Mean ± SD |
| AUC₀₋ₜ (ng · hr/mL) | 1204.14 ± 469.11 | 1027.09 ± 328.02 |
| Cₘₐₓ (ng/mL) | 39.40 ± 14.82 | 29.28 ± 7.16 |
| Tₘₐₓ (hr) | 16.00 ± 4.07 | 13.87 ± 4.93 |
| | (16.00*) | (12.00*) |
| Aₑ (mg) | 9.02 ± 2.72 | 8.29 ± 2.94 |
| CLᵣ (L/hr) | 8.05 ± 2.28 | 8.40 ± 2.64 |

II. Summary of bioavailability assessments for N,O-
didesmethylvenlafaxine in plasma (Treatment A v. Treatment B)

| | | Geometric Mean |
|---|---|---|
| | 90% Confidence Interval | Ratio |
| AUC₀₋ₜ | 106-122 | 1.15 |
| Cₘₐₓ | 115-141 | 1.28 |

*Median Value

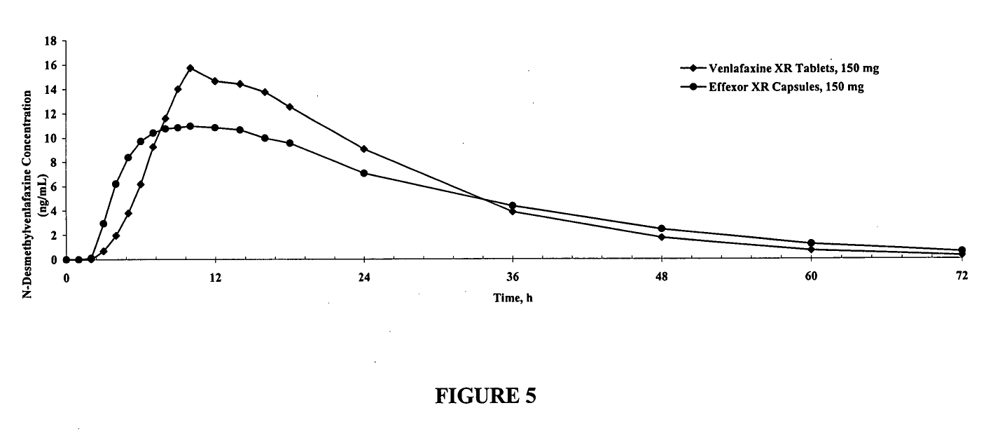

The blood-plasma concentration time curve for DDV from the composition of the invention versus that from Effexor® XR is shown in FIG. 5.

TABLE 9
(N-DESMETHYLVENLAFAXINE)
I. Summary of pharmacokinetic results for N-desmethylvenlafaxine
in plasma and urine

| Pharmacokinetic | Treatment A | Treatment B |
|---|---|---|
| Parameters | Mean ± SD | Mean ± SD |
| AUC₀₋ₜ (ng · hr/mL) | 365.60 ± 439.74 | 340.16 ± 432.41 |
| Cₘₐₓ (ng/mL) | 17.53 ± 16.68 | 12.26 ± 11.31 |
| Tₘₐₓ (hr) | 11.60 ± 2.92 | 8.80 ± 3.57 |
| | (10.00*) | (8.00*) |
| Aₑ (mg) | 2.34 ± 2.70 | 2.21 ± 2.95 |
| CLᵣ (L/hr) | 5.69 ± 1.51 | 5.73 ± 3.07 |

II. Summary of bioavailability assessments for N-desmethylvenlafaxine
in plasma (Treatment A v. Treatment B)

| | | Geometric Mean |
|---|---|---|
| | 90% Confidence Interval | Ratio |
| AUC₀₋ₜ | 94-138 | 1.15 |
| Cₘₐₓ | 103-153 | 1.27 |

*Median Value

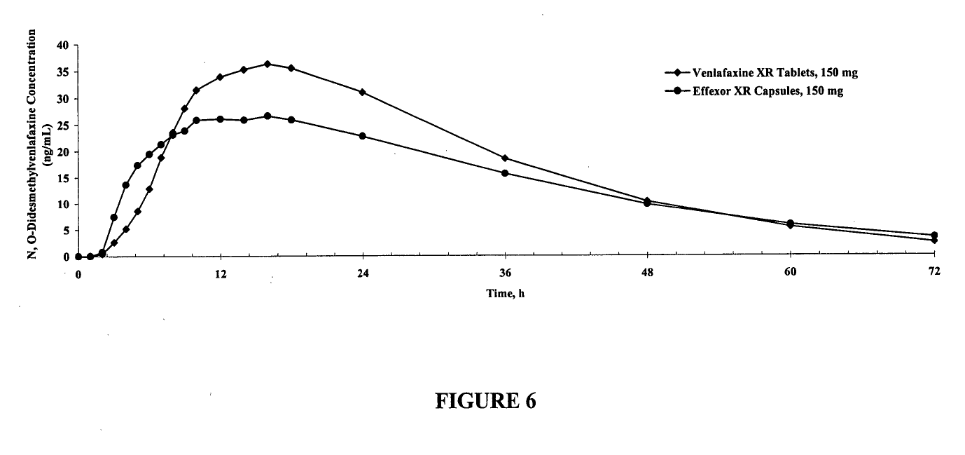

The blood-plasma concentration time curve for NDV from the composition of the invention versus that from Effexor® XR is shown in FIG. 6.

TABLE 10
(N,N,O-TRIDESMETHYLVENLAFAXINE)
I. Summary of pharmacokinetic results for N-desmethylvenlafaxine
in plasma and urine

| Pharmacokinetic | Treatment A | Treatment B |
|---|---|---|
| Parameters | Mean ± SD | Mean ± SD |
| AUC₀₋ₜ (ng · hr/mL) | 163.52 ± 139.97 | 146.38 ± 127.15 |
| Cₘₐₓ (ng/mL) | 4.97 ± 2.86 | 3.78 ± 2.08 |
| Tₘₐₓ (hr) | 19.29 ± 6.45 | 17.14 ± 7.79 |
| | (17.00*) | (17.00*) |
| Aₑ (mg) | 1.78 ± 1.28 | 1.62 ± 1.40 |
| CLᵣ (L/hr) | 11.90 ± 4.63 | 12.08 ± 7.12 |

II. Summary of bioavailability assessments for N,N,O-Tridesmethyl-
venlafaxine in plasma (Treatment A v. Treatment B)

| | | Geometric Mean |
|---|---|---|
| | 90% Confidence Interval | Ratio |
| AUC₀₋ₜ | 98-142 | 1.12 |
| Cₘₐₓ | 113-138 | 1.28 |

*Median Value

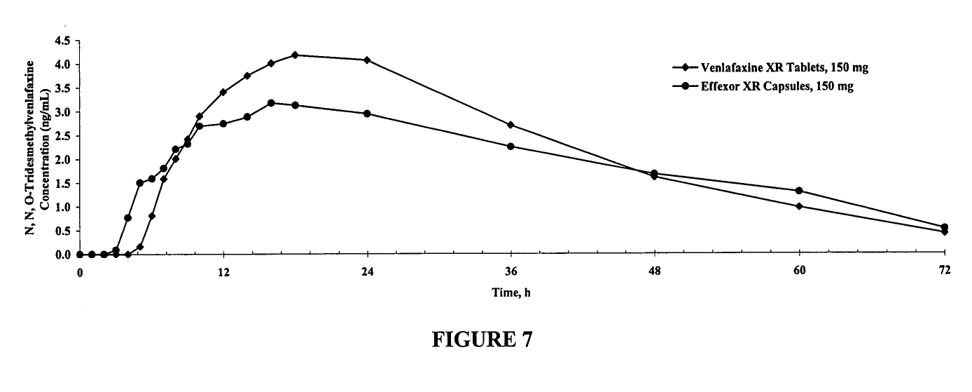

The blood-plasma concentration time curve for TDV from the composition of the invention versus that from Effexor® XR is shown in FIG. 7.

Table 11 summarizes the metabolite-parent AUC ratio in the fed state:

TABLE 11

| | ODV | NDV | DDV | TDV |
|---|---|---|---|---|
| Venlafaxine HCl XR Tablets 150 mg | 2.538 | 0.176 | 0.579 | 0.073 |
| Effexor ® XR Capsules 150 mg | 2.379 | 0.183 | 0.551 | 0.073 |

The incidence of adverse events resulting from the two treatments for the fasting study are tabulated in Table 12:

TABLE 12

| | Venlafaxine HCL XR | Effexor ® XR Capsules, |
|---|---|---|
| Adverse Event | Tablets, 150 mg (n = 13) | 150 mg (n = 14) |
| Any adverse event | 6 (46%) | 8 (57%) |
| Nausea | 2 (15%) | 4 (29%) |
| Vomiting | 0 | 2 (14%) |
| Dizziness | 1 (7.7%) | 4 (29%) |
| Muscle Tightness | 3 (23%) | 2 (14%) |
| Tremor | 1 (7.7%) | 0 |
| Feeling Cold | 0 | 1 (7.1%) |
| Headache | 0 | 1 (7.1%) |
| Insomnia | 0 | 2 (14%) |
| Dry Mouth | 1 (7.1%) | 1 (7.1%) |
| Somnolence | 0 | 2 (14%) |

### EXAMPLE 3B

The objective of this study was to compare the peak and systemic exposure of venlafaxine and its metabolites from a test formulation of venlafaxine hydrochloride 150 mg tablets of the invention versus the reference Effexor® XR 150 mg capsules under fasting conditions. Bioavailability of these formulations was assessed for venlafaxine, ODV, NDV, DDV, and TDV. In addition, urinary recovery of venlafaxine, ODV, NDV, DDV, TDV, ODV glucuronide and DDV glucuronide was compared from the two products.

The study design involved a two-way, crossover, open-label single-dose, fasting, bioavailability study of 150 mg venlafaxine hydrochloride tablets of the invention versus the reference-product Effexor® XR 150 mg capsules in normal healthy non-smoking male subjects. Sixteen normal, healthy, non-smoking male subjects, within an age range of 18 to 65 years were selected the study after meeting several inclusion and exclusion criteria no more than 30 days prior to the first drug administration. All subjects underwent a medical history, medication history, physical examination (including blood pressure, heart rate and temperature) and ECG prior to starting the study. Selected routine clinical laboratory measurements, including screens for hepatitis C, hepatitis B-surface antigen, HIV, urine drugs of abuse, cotinine and saliva alcohol were performed during the screening. At check-in for each study period, screens for urine drugs of abuse, cotinine and saliva alcohol were performed on all subjects. There were 15 subjects dosed in period I, 14 of whom completed the study. One subject who experienced vomiting within twice the median Tₘₐₓ was excluded from the statistical analysis as per FDA guidelines. Therefore, pharmacokinetic and statistical analyses were performed on 13 of the 14 subjects who completed the study.

Subjects received one of the following treatments at 0.0 hour on Day 1 of each study period according to a computer generated randomization scheme:

Treatment A

One test formulation of venlafaxine hydrochloride XR 150 mg tablet of the invention administered with 240 ml of ambient temperature water following an overnight fast of at least ten hours.

Treatment B

One Effexor® XR 150 mg capsule administered with 240 ml ambient temperature water following an overnight fast of at least 10 hours (Treatment Dose=150 mg).

The study consisted of two 4-day study periods separated by at least a two-week washout period between treatments.

Water was provided ad libitum until 1.0 hour pre-dose. For both treatments, except during the first hour post-dose, when 100 ml of ambient temperature water was administered at 1.0 hour, 150 ml of ambient temperature water was administered according to the following schedule: 2.0, 3.0, 4.0, 5.0, 6.0, 7.0. 8.0, 9.0, 10.0, 11.0, 12.0, 16.0, 20.0, 24.0, 28.0, 32.0, 36.0, 40.0, 44.0, 48.0, 52.0, 56.0, 60.0, 64.0 and 68.0 hours post-dose. After 68.0 hours post drug administration, water was permitted ad libitum. No additional fluid intake was permitted outside of the scheduled time points, except for the 240 ml of water used to administer the study drug and the additional fluid provided with meals. Each serving of water must be consumed within 5 minutes. In instances where time to void and time of water administration coincide, the subject was asked to void prior to consuming water.

Twenty blood samples (7 ml each) were drawn in each period for each treatment according to the following schedule: 0.0 (pre-dose), 1.0,2.0, 3.0, 4.0, 5.0, 6.0, 7.0, 8.0, 9.0, 10.0, 12.0, 14.0, 16.0, 18.0, 24.0, 36.0, 48.0, 60.0 and 72.0 hours post-drug administration.

Urine was collected from all subjects during the following time intervals: Prior to dosing (complete void and collect), 0.0-2.0, 2.0-4.0, 4.0-8.0, 8.0-12.0, 12.0-24.0, 24.0-48.0 and 48.0-72.0 hours post-dose. For each individual subject, all urine samples for each specified time interval were collected and pooled. A minimum of 10 ml of urine was required for each time interval for each subject. For each time interval, the pH and volume of the pooled urine sample was measured and recorded.

The following pharmacokinetic parameters for venlafaxine, ODV, NDV, DDV, and TDV were calculated by standard non-compartmental methods: AUC₀₋ₜ, Cₘₐₓ, Tₘₐₓ, Clᵣ, Aₑ, and AUCₘₑₜ/AUCₚₐᵣₑₙₜ.

Descriptive statistics were performed for plasma and urine concentrations and for all PK parameters. Using GLM procedures in SAS, analysis of variance (ANOVA) was performed on Log-transformed AUC₀₋ₜ, and Cₘₐₓ and on untransformed Tₘₐₓ, Clᵣ, Aₑ, AUCₘₑₜ/AUCₚₐᵣₑₙₜ at a significance level of 0.05. The intra-subject coefficient of variation (CV) was calculated using the Mean Square Error (MSE) from the ANOVA table. The ratio of geometric means and the 90% geometric confidence interval (90% C.I.) were calculated based on the difference in the Least Squares Means of the log-transformed AUC₀₋ₜ, and Cₘₐₓ between the test and reference formulations.

The incidences of all adverse events were tabulated by treatment group and subject number. MedDRA Version 6.1 was used to document all Adverse Events.

Tables 13-19 summarize data obtained for this study:

TABLE 13
(VENLAFAXINE)
I. Summary of pharmacokinetic results for venlafaxine in plasma and urine

| Pharmacokinetic | Treatment A | Treatment B |
|---|---|---|
| Parameters | Mean ± SD | Mean ± SD |
| AUC₀₋ₜ (ng · hr/mL) | 1987.49 ± 1506.75 | 162.77 ± 1205.24 |
| Cₘₐₓ (ng/mL) | 107.27 ± 56.32 | 195.48 ± 41.30 |
| Tₘₐₓ (hr) | 10.77 ± 2.39 | 7.92 ± 3.38 |
| | (10.00*) | (8.00*) |
| Aₑ (mg) | 13.646 ± 11.458 | 10.947 ± 9.027 |
| CLᵣ (L/hr) | 7.190 ± 2.069 | 6.87 ± 2.668 |

II. Summary of bioavailability assessments for venlafaxine in plasma
(Treatment A v. Treatment B)

| | | Geometric Mean |
|---|---|---|
| | 90% Confidence Interval | Ratio |
| AUC₀₋ₜ | 101-131 | 1.15 |
| Cₘₐₓ | 98-122 | 1.29 |

*Median Value

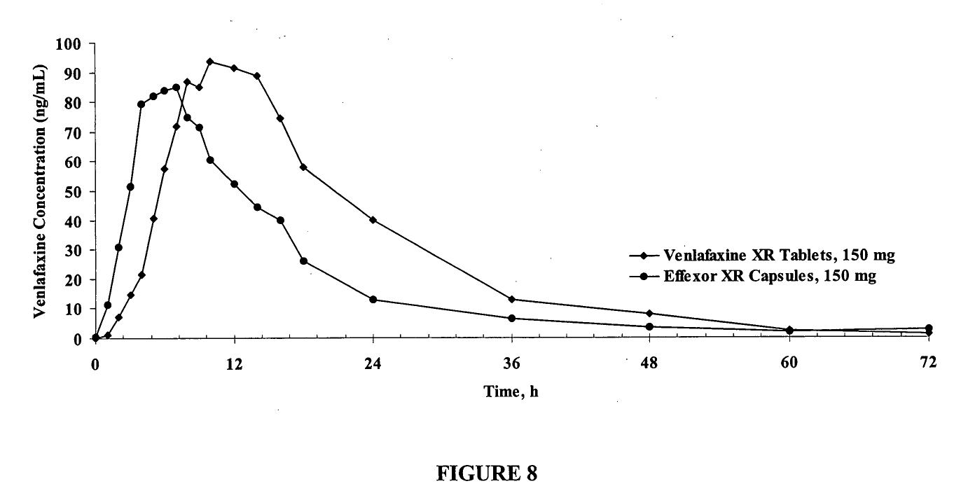

The blood-plasma concentration time curve for venlafaxine from the composition of the invention versus that from Effexor® XR is shown in FIG. 8.

TABLE 14
(O-DESMETHYLVENLAFAXINE)
I. Summary of pharmacokinetic results for
O-desmethylvenlafaxine in plasma and urine

| Pharmacokinetic | Treatment A | Treatment B |
|---|---|---|
| Parameters | Mean ± SD | Mean ± SD |
| AUC₀₋ₜ (ng · hr/mL) | 4499.84 ± 1277.73 | 4133.94 ± 1057.61 |
| Cₘₐₓ (ng/mL) | 167.14 ± 51.61 | 150.80 ± 48.49 |
| Tₘₐₓ (hr) | 14.15 ± 2.38 | 12.54 ± 2.85 |
| | (14.00*) | (12.00*) |
| Aₑ (mg) | 40.70 ± 13.99 | 34.67 ± 11.19 |
| CLᵣ (L/hr) | 9.18 ± 2.23 | 8.59 ± 2.49 |

II. Summary of bioavailability assessments for
O-desmethylvenlafaxine in plasma (Treatment A v. Treatment B)

| | | Geometric Mean |
|---|---|---|
| | 90% Confidence Interval | Ratio |
| AUC₀₋ₜ | 99-115 | 1.18 |
| Cₘₐₓ | 100-120 | 1.09 |

*Median Value

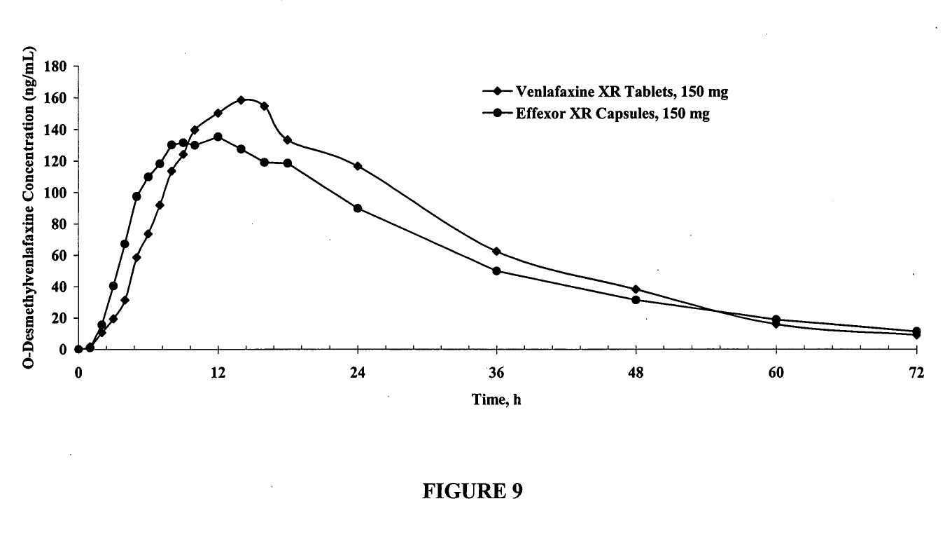

The blood-plasma concentration time curve for ODV from the composition of the invention versus that from Effex® XR is shown in FIG. 9.

TABLE 15
(N,O-DIDESMETHYLVENLAFAXINE)
I. Summary of pharmacokinetic results for
N,O-didesmethylvenlafaxine in plasma and urine

| Pharmacokinetic | Treatment A | Treatment B |
|---|---|---|
| Parameters | Mean ± SD | Mean ± SD |
| AUC₀₋ₜ (ng · hr/mL) | 963.76 ± 392.76 | 854.08 ± 279.78 |
| Cₘₐₓ (ng/mL) | 27.70 ± 10.52 | 25.54 ± 7.03 |
| Tₘₐₓ (hr) | 17.85 ± 7.01 | 14.77 ± 4.57 |
| | (16.00*) | (16.00*) |
| Aₑ (mg) | 9.956 ± 3.618 | 8.282 ± 2.513 |
| CLᵣ (L/hr) | 10.862 ± 2.260 | 10.123 ± 2.538 |

II. Summary of bioavailability assessments for
N,O-didesmethylvenlafaxine in plasma (Treatment A v. Treatment B)

| | | Geometric Mean |
|---|---|---|
| | 90% Confidence Interval | Ratio |
| AUC₀₋ₜ | 95-119 | 1.06 |
| Cₘₐₓ | 86-127 | 1.04 |

*Median Value

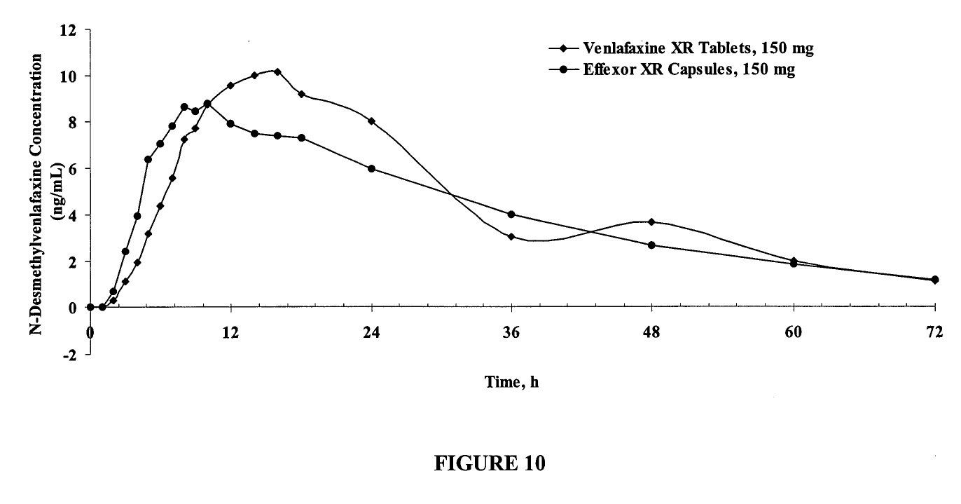

The blood-plasma concentration time curve for DDV from the composition of the invention versus that from Effexor® XR is shown in FIG. 10.

TABLE 16
(N-DESMETHYLVENLAFAXINE)
I. Summary of pharmacokinetic results for
N-desmethylvenlafaxine in plasma and urine

| Pharmacokinetic | Treatment A | Treatment B |
|---|---|---|
| Parameters | Mean ± SD | Mean ± SD |
| AUC₀₋ₜ (ng · hr/mL) | 361.50 ± 540.64 | 301.43 ± 495.56 |
| Cₘₐₓ (ng/mL) | 11.50 ± 11.83 | 10.31 ± 9.58 |
| Tₘₐₓ (hr) | 14.67 ± 7.65 | 9.77 ± 4.68 |
| | (13.00*) | (8.00*) |
| Aₑ (mg) | 3.76 ± 5.97 | 2.68 ± 4.40 |
| CLᵣ (L/hr) | 7.61 ± 2.29 | 7.85 ± 3.17 |

II. Summary of bioavailability assessments for
N-desmethylvenlafaxine in plasma (Treatment A v. Treatment B)

| | | Geometric Mean |
|---|---|---|
| | 90% Confidence Interval | Ratio |
| AUC₀₋ₜ | 96.5-163 | 1.25 |
| Cₘₐₓ | 80-133 | 1.03 |

*Median Value

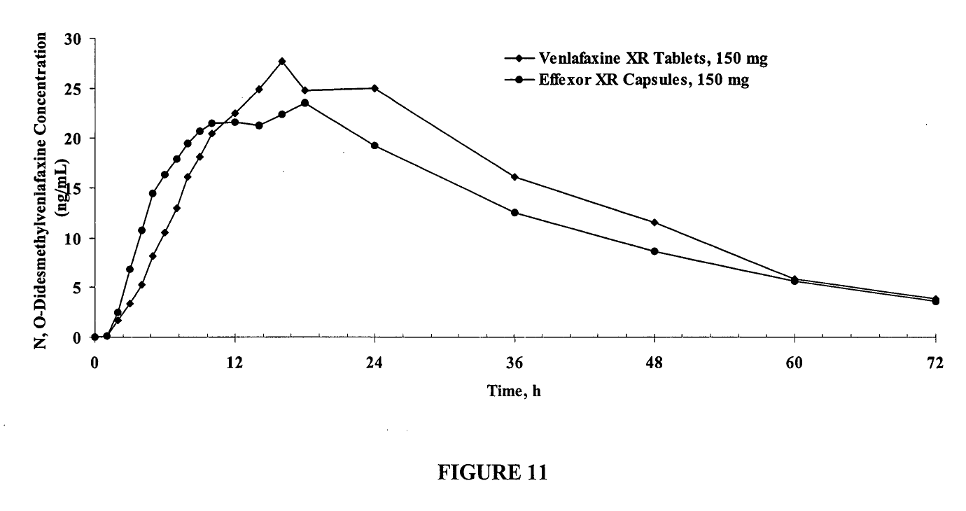

The blood-plasma concentration time curve for NDV from the composition of the invention versus that from Effexor® XR is shown in FIG. 11.

TABLE 17
(N,N,O-DESMETHYLVENLAFAXINE)
I. Summary of pharmacokinetic results for
N-desmethylvenlafaxine in plasma and urine

| Pharmacokinetic | Treatment A | Treatment B |
|---|---|---|
| Parameters | Mean ± SD | Mean ± SD |
| AUC₀₋ₜ (ng · hr/mL) | 168.98 ± 242.31 | 157.91 ± 278.97 |
| Cₘₐₓ (ng/mL) | 4.37 ± 4.77 | 4.03 ± 4.71 |
| Tₘₐₓ (hr) | 20.84 ± 6.63 | 18.50 ± 11.82 |
| | (21.00*) | (16.00*) |
| Aₑ (mg) | 2.23 ± 2.75 | 1.62 ± 2.68 |
| CLᵣ (L/hr) | 11.57 ± 5.81 | 11.71 ± 5.39 |

II. Summary of bioavailability assessments for
N,N,O-desmethylvenlafaxine in plasma (Treatment A v. Treatment B)

| | | Geometric Mean |
|---|---|---|
| | 90% Confidence Interval | Ratio |
| AUC₀₋ₜ | 68-158 | 1.03 |
| Cₘₐₓ | 81-145.5 | 1.09 |

*Median Value

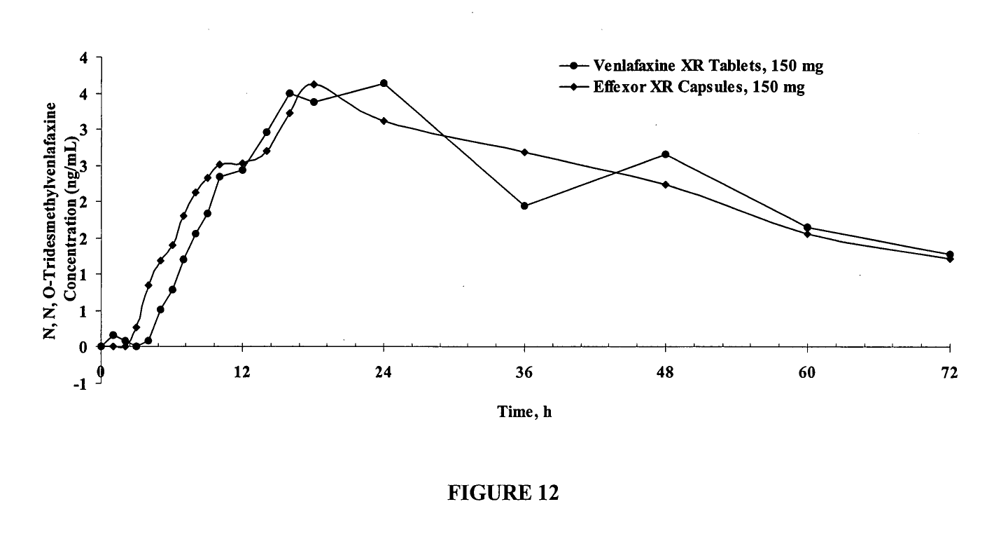

The blood-plasma concentration time curve for TDV from the composition of the invention versus that from Effexor® XR is shown in FIG. 12.

Table 17 summarizes the metabolite-parent AUC ratio in the fasted state:

TABLE 18

| | ODV | NDV | DDV | TDV |
|---|---|---|---|---|
| Venlafaxine HCl XR Tablets 150 mg | 4.145 | 0.194 | 0.749 | 0.179 |
| Effexor ® XR Capsules 150 mg | 4.341 | 0.162 | 0.841 | 0.133 |

The incidence of adverse events resulting from the two treatments for the fed study are tabulated in Table 19:

TABLE 19

| | | Effexor ® XR |
|---|---|---|
| | Venlafaxine HCL XR | Capsules, |
| Adverse Event | Tablets, 150 mg (n = 13) | 150 mg (n = 14) |
| Any adverse event | 5 (33%) | 5 (33%) |
| Nausea | 2 (13%) | 3 (20%) |
| Dizziness | 1 (6.7%) | 1 (6.7%) |
| Muscle Tightness | 1 (6.7%) | 1 (6.7%) |
| Hiccups | 1 (6.7%) | 0 |
| Fatigue | 1 (6.7%) | 0 |
| Headache | 1 (6.7%) | 0 |
| Increased blood pressure | 0 | 1 (6.7%) |
| Loose stool | 1 (6.7%) | 0 |
| Somnolence | 0 | 1 (6.7%) |

### EXAMPLE 4

Three core formulations were made as shown in Table 20:

TABLE 20

| | Core | | Core | | Core | |
|---|---|---|---|---|---|---|
| | Formulation E | | Formulation F | | Formulation G | |
| | Quantity | | Quantity | | Quantity | |
| Ingredient | (mg) | % w/w | (mg) | % w/w | (mg) | % w/w |
| Venlafaxine HCl | 42.43 | 54.051 | 42.43 | 53.04 | 42.43 | 53.04 |
| Gelling agent¹ | 17.5 | 22.293 | — | — | — | — |
| Filler² | — | — | 19 | 23.75 | 19 | 23.75 |
| Binder³ | 45 | 5.732 | 5 | 6.25 | 5 | 6.25 |
| Gelling agent⁴ | 12.5 | 15.924 | 12 | 15 | 12 | 15 |
| Lubricant⁵ | 1.57 | 2 | 1.57 | 1.96 | 1.57 | 1.96 |
| Solvent⁶(ml) | 28 | — | 28 | — | 22 | — |
| Core tablet weight (mg) | 78.5 | 100 | 80 | 100 | 80 | 100 |

¹Xantural 180
²Lactose # 315 Spray Dried
³Plasdone K29/32 (PVP)
⁴Methocel Premium E3 LV
⁵Sodium Stearyl Fumarate N.F.
⁶Isopropyl Alcohol 99% USP. Evaporates after drying.

The cores were made as described in Example 1.

### EXAMPLE 5

The cores of Example 5 were coated with the following coat formulations shown in Table 21:

TABLE 21

| | Coat | | Coat | | Coat | | Coat | |
|---|---|---|---|---|---|---|---|---|
| | Formulation H | | Formulation I | | Formulation J | | Formulation K | |
| | Quantity | | Quantity | | Quantity | | Quantity | |
| Ingredient | (mg) | % w/w | (mg) | % w/w | (mg) | % w/w | (mg) | % w/w |
| Water-insoluble water- | 7.82 | 55.86 | 7.64 | 54.57 | 6.7 | 58.26 | 7.29 | 58.32 |
| permeable film forming | | | | | | | | |
| polymer¹ | | | | | | | | |
| Water-soluble polymer² | 3.85 | 27.5 | 4.03 | 28.79 | 2.88 | 25.04 | 3.13 | 25.04 |
| Plasticizer³ | 2.33 | 16.64 | 2.33 | 16.64 | 1.92 | 16.7 | 2.08 | 16.64 |
| Solvent⁴ | 134.48 | — | 134.48 | — | 110.46 | — | 120.07 | — |
| Solvent⁵ | 7.08 | — | 7.08 | — | 5.8 | — | 6.32 | — |
| Total Weight | 155.56 | — | 155.56 | — | 127.76 | — | 138.89 | — |
| Total dry solids | 14 | 100 | 14 | 100 | 11.5 | 100 | 12.5 | 100 |
| Coated Tablet weight | 92.5 | | 92.5 | | 91.5 | | 92.5 | |
| (mg) | | | | | | | | |
| Total weight in capsule | 370 | | 370 | | 366 | | 370 | |
| Concentration of solids | 9 | | 9 | | 9 | | 9 | |
| (% w/w) | | | | | | | | |

¹Ethocel 100 STD Premium
²Kollidon 90F
³Dibutyl Sebacate NF
⁴Ethyl alcohol 200 Proof Evaporates after drying.
⁵Isopropyl alcohol 99% USP. Evaporates after drying.

The cores were coated as described in Example 2. Core Formulation E was coated with Coat Formulation H or I, Core Formulation F was coated with Coat Formulation J and Core Formulation G was coated with Coat Formulation K.

The dissolution of the coated tablets was determined under the same experimental conditions as described in Example 1. The results are presented in Table 22 as % released of the total venlafaxine hydrochloride in the coated tablet cores:

TABLE 22

| | Core Formulation | Core Formulation E | Core Formulation | Core Formulation | |
|---|---|---|---|---|---|
| | E with Coat | with Coat | F with Coat | G with Coat | |
| Time | Formulation H | Formulation I | Formulation J | Formulation K | |
| (hr) | (Formulation EH) | (Formulation EI) | (Formulation FJ) | (Formulation GK) | Effexor |
| 0 | 0.0 | 0.0 | 0.0 | 0.0 | 0.0 |
| 1 | 2.9 | 4.4 | 1.3 | 0.8 | 4.0 |
| 2 | 10.8 | 16.0 | 5.3 | 4.1 | 16.0 |
| 3 | 21.8 | 30.4 | 11.0 | 8.7 | 31.0 |
| 4 | 34.4 | 45.0 | 18.0 | 14.3 | 45.0 |
| 5 | 46.8 | 57.9 | 26.1 | 20.9 | 55.0 |
| 6 | 58.2 | 68.0 | 36.6 | 28.7 | 62.0 |
| 7 | 67.5 | 75.2 | 48.0 | 37.5 | 68.0 |
| 8 | 74.7 | 79.5 | 58.8 | 46.8 | 72.0 |
| 9 | 79.9 | 81.7 | 68.7 | 56.0 | 76.0 |
| 10 | 83.2 | 82.8 | 77.1 | 64.7 | 79.0 |
| 11 | 85.0 | 83.5 | 83.8 | 72.4 | 81.0 |
| 12 | 86.0 | 84.2 | 88.9 | 78.9 | 84.0 |
| 13 | 86.7 | 85.0 | 92.8 | 84.2 | 85.0 |
| 14 | 87.3 | 85.8 | 95.6 | 88.4 | 87.0 |
| 15 | 87.8 | 86.8 | 97.6 | 91.7 | 89.0 |
| 16 | 88.4 | 87.9 | 98.9 | 94.3 | 90.0 |
| 17 | 89.1 | 89.5 | 99.9 | 96.1 | 91.0 |
| 18 | 89.8 | 91.1 | 100.7 | 97.4 | 93.0 |
| 19 | 90.6 | 92.8 | 101.2 | 98.4 | 94.0 |
| 20 | | 95.7 | 101.5 | 99.1 | 95.0 |

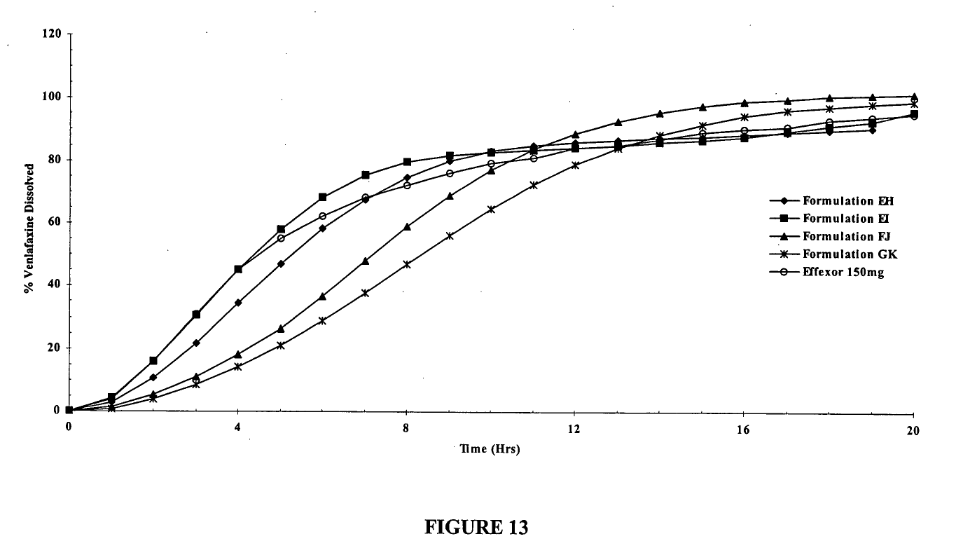

The data is graphically presented in FIG. 13.

Dissolution of coated tablet cores shown in Table 22 was also determined under the following dissolution conditions:
Medium: pH 6.8 buffer
Method: USP Type I apparatus, 75 rpm at 37° C.

The results are shown in Table 23 and are presented as % released of the total venlafaxine hydrochloride in the tablet cores:

TABLE 23

| Time | | Formulation | Formulation | Formulation | Formulation |
|---|---|---|---|---|---|
| (hr) | Effexor | EH | EI | FJ | GK |
| 0 | 0.0 | 0.0 | 0.0 | 0.0 | 0.0 |
| 1 | 6.4 | 2.0 | 3.6 | 0.6 | 0.5 |
| 2 | 18.6 | 9.0 | 13.8 | 3.7 | 3.6 |
| 3 | 33.4 | 19.2 | 27.1 | 8.4 | 8.2 |
| 4 | 46.1 | 30.7 | 40.6 | 14.4 | 13.9 |
| 5 | 55.6 | 42.3 | 52.7 | 21.8 | 20.7 |
| 6 | 62.7 | 52.8 | 62.6 | 30.1 | 28.6 |
| 7 | 68.3 | 61.7 | 70.4 | 39.6 | 37.2 |
| 8 | 72.7 | 68.9 | 76.5 | 50.5 | 46.2 |
| 9 | 76.4 | 74.8 | 81.3 | 60.6 | 54.8 |
| 10 | 79.4 | 79.7 | 85.2 | 69.6 | 62.7 |
| 11 | 82.1 | 83.6 | 88.3 | 77.1 | 69.7 |
| 12 | 84.3 | 87.0 | 90.9 | 83.6 | 75.7 |
| 13 | 86.4 | 89.8 | 93.1 | 89.1 | 80.7 |
| 14 | 88.2 | 92.4 | 94.8 | 93.3 | 85.0 |
| 15 | 89.8 | 94.4 | 96.3 | 97.3 | 88.5 |
| 16 | 91.3 | 96.2 | 97.5 | 100.2 | 91.3 |
| 17 | — | 97.8 | 98.5 | 102.8 | 93.7 |
| 18 | — | 99.2 | 99.4 | 104.9 | 95.8 |
| 19 | — | 100.5 | 100.1 | 106.8 | 97.4 |
| 20 | — | 101.6 | 100.7 | 106.0 | 98.8 |

WEIBULL PARAMETERS

| | | Formulation | Formulation | Formulation | Formulation |
|---|---|---|---|---|---|
| | Effexor | EH | EI | FJ | GK |
| A | 0.119 | 0.040 | 0.066 | 0.008 | 0.009 |
| B | 1.134 | 1.601 | 1.477 | 2.182 | 2.031 |

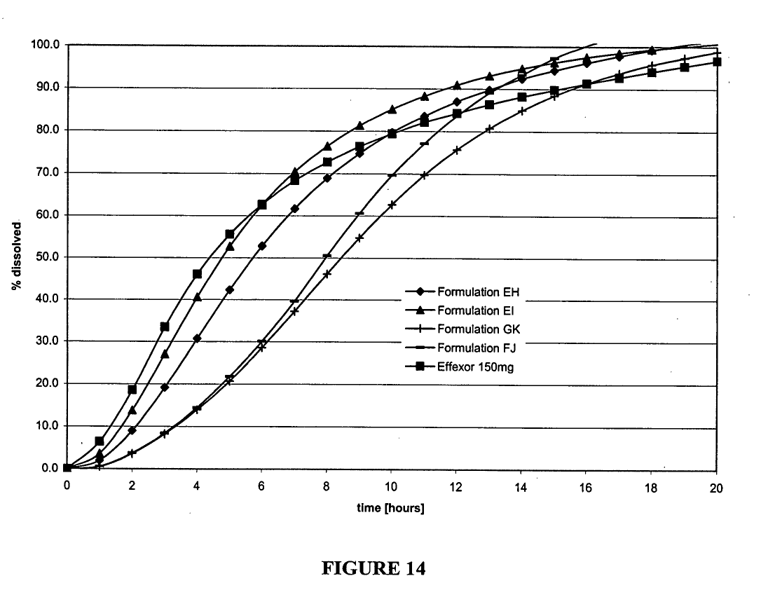

The data is graphically presented in FIG. 14.

### EXAMPLE 6

Pharmacokinetic Studies

The objective of this study was to compare the peak and systemic exposure of venlafaxine from Formulations EH, EI, FJ and GK versus the reference Effex® XR 150 mg capsules under fasting conditions. Bioavailability of these formulations was assessed for venlafaxine.

The study design followed the study design described in Example 3B under fasting conditions. Table 24 summarizes the data obtained for this study:

TABLE 24

| | | | AUC | | C_{MAX} | | |
|---|---|---|---|---|---|---|---|
| Formulation | N | AUC_{(0-T)} | RATIO | C_{MAX} | RATIO | T_{MAX}* | T_{LAG} |
| FJ | 9 | 1344.24 ± 398.74 | 132.2 | 86.37 ± 30.60 | 121.80 | 10.7 | 3.78 ± 0.44 |
| GK | 9 | 1298.02 ± 336.39 | 128.6 | 73.80 ± 19.50 | 107.0 | 11.8 | 4.0 ± 0.0 |
| Effexor | 9 | 1012.76 ± 272.48 | | 69.77 ± 22.20 | | 6.70 | 3.22 ± 0.44 |
| EH | 9 | 1565.69 ± 806.58 | 136.90 | 107.51 ± 41.1 | 146.50 | 8.0 | 3.11 ± 0.33 |
| EI | 9 | 1615.02 ± 976.70 | 136.40 | 114.72 ± 54.4 | 154.40 | 8.0 | 3.11 ± 0.33 |
| Effexor | 9 | 1127.00 ± 496.59 | | 71.60 ± 21.6 | | 7.0 | 3.0 ± 0.00 |

Conclusion

The present invention relates to a modified release composition for oral administration of at least one form of venlafaxine. In particular, the present invention relates to an enhanced absorption delayed controlled release composition of at least one form of venlafaxine.

As demonstrated by the geometric mean ratios, the enhanced absorption delayed controlled-release composition of the invention demonstrated higher bioavailability and higher mean peak plasma concentrations of venlafaxine and its active metabolite ODV when compared to the reference product, Effexor® XR 150 mg capsule, under single-dose fasting or fed conditions. For example, the individual geometric mean ratio (GMR) of the composition of the invention to the reference product Effexor® XR when administered under fed or fasting conditions as a single dose for the AUC₀₋ₜ for venlafaxine or its active metabolite ODV is greater than 1. Similarly, the individual GMR of the composition of the invention to the reference product for the Cₘₐₓ for venlafaxine or its active metabolite is also greater than 1. The combined GMR (GMR_{c}), which is the individual GMR for venlafaxine plus the individual GMR for ODV, for the AUC₀₋ₜ or for the Cₘₐₓ under fed or fasting conditions is greater than 2.

More specifically, under fed conditions the individual GMR for the AUC₀₋ₜ and for the Cₘₐₓ for venlafaxine is about 1.14 and about 1.29 respectively. For ODV, the individual GMR for the AUC₀₋ₜ and for the Cₘₐₓ is about 1.18 and 1.35 respectively. The GMR_{c} for the AUC₀₋ₜ and for Cₘₐₓ is 2.32 and 2.65 respectively. Further, the composition of the invention compared to the reference product exhibits a delay in the Tₘₐₓ of about 5 hours for venlafaxine and about 2 hours for ODV.

Under fasting conditions the individual GMR for the AUC₀₋ₜ and for the Cₘₐₓ for venlafaxine is about 1.15 and about 1.29 respectively. For ODV, the individual GMR for the AUC₀₋ₜ and for the Cₘₐₓ is about 1.18 and 1.09 respectively. The GMR_{c} for the AUC₀₋ₜ and for Cₘₐₓ is 2.33 and 2.38 respectively. Further, the composition of the invention compared to the reference product exhibits a delay in the Tₘₐₓ of about 2 hours for both venlafaxine and ODV.

One consequence of the enhanced absorption character of the composition of the invention is that the urinary recovery of venlafaxine and its four metabolites was also larger after oral administration of the composition of the invention.

Despite the higher AUC₀₋ₜ and Cₘₐₓ of venlafaxine and ODV for the composition of the invention, the side effects resulting from administration of the enhanced absorption delayed controlled release composition of the invention is similar or less than the adverse events observed after administration of the reference product, Effexor® XR, 150 mg capsules. As stated above, it is believed that this is achieved because of the release profile, which conforms to the Weibull distribution described herein. Moreover, the adverse events observed with the composition of the invention are not influenced by food. A further advantage of the enhanced absorption delayed controlled-release composition of the invention is the potential of decreasing the absolute amount of the at least one form of venlafaxine comprising the core to an amount that is less than the absolute amount of active in the reference product Effexor® XR, 150 mg capsules. Accordingly, such a composition could be made bioequivalent to the reference product and result in an even better safety profile compared to the reference product.

1-82. (canceled)

83. An enhanced absorption delayed controlled release pharmaceutical composition for oral administration suitable for once daily dosing comprising:
a) a core comprising at least one form of venlafaxine selected from the group consisting of venlafaxine, a pharmaceutically acceptable salt of venlafaxine, an active metabolite of venlafaxine, a pharmaceutically acceptable salt of an active metabolite of venlafaxine, and combinations thereof, and pharmaceutically acceptable excipient; and
b) a coating substantially surrounding said core, said coating comprising a water-insoluble water-permeable film-forming polymer, a water-soluble polymer or substance, and a plasticizer,
wherein said composition provides enhanced absorption delayed controlled release of said at least one form of venlafaxine such that the combined geometric mean ratio of the composition of the invention to the reference product for the AUC₀₋ₜ for venlafaxine and its active metabolite O-desmethylvenlafaxine is greater than 1 after first administration under fed or fasting conditions.

84. The composition of claim 83, wherein said combined geometric mean ratio for the AUC₀₋ₜ is about 2.32 under fed conditions.

85. The composition of claim 83, wherein said combined geometric mean ratio for the AUC₀₋ₜ is about 2.33 under fasting conditions.

86. The composition of claim 83, wherein said combined geometric mean ratio for the Cₘₐₓ is about 2.65 under fed conditions.

87. The composition of claim 83, wherein said combined geometric mean ratio for the Cₘₐₓ is about 2.38 under fasting conditions.

88. The composition of claim 83, wherein the Tₘₐₓ of the composition compared to the reference product for venlafaxine is delayed by about 5 hours under fed conditions.

89. The composition of claim 83, wherein the Tₘₐₓ of the composition compared to the reference product for O-desmethylvenlafaxine is delayed by about 2 hours under fed conditions.

90. The composition of claim 83, wherein the Tₘₐₓ of the composition compared to the reference product for venlafaxine is delayed by about 2 hours under fasting conditions.

91. The composition of claim 83, wherein the Tₘₐₓ of the composition compared to the reference product for O-desmethylvenlafaxine is delayed by about 2 hours under fasting conditions.

92. The composition of claim 83, wherein said composition provides a Tₘₐₓ for venlafaxine and O-desmethylvenlafaxine greater than about 8 hours after administration of the composition in the fed or fasted state.

93. The composition of claim 83, wherein said composition provides a Tₘₐₓ for venlafaxine at about 11 hours after administration of the composition in the fed state.

94. The composition of claim 83, wherein said composition provides a Tₘₐₓ for 0-desmethylvenlafaxine at about 12 hours after administration of the composition in the fed state.

95. The composition of claim 83, wherein said composition provides a Tₘₐₓ for venlafaxine at about 10 hours after administration of the composition in the fasted state.

96. The composition of claim 83, wherein said composition provides a Tₘₐₓ for O-desmethylvenlafaxine at about 14 hours respectively in the fasted state.

97. The composition of claim 83, wherein said composition provides a Cₘₐₓ greater than 150 ng/ml for venlafaxine and O-desmethylvenlafaxine in the fed state.

98. The composition of claim 83, wherein said composition provides a Cₘₐₓ of about 160 ng/ml for venlafaxine in the fed state.

99. The composition of claim 83, wherein said composition provides a Cₘₐₓ of about 211 ng/ml for O-desmethylvenlafaxine in the fed state.

100. The composition of claim 83, wherein said composition provides an in vitro dissolution profile using the USP Type I apparatus method at 75 rpm in 1000 ml phosphate buffer pH 6.8 at 37° C. characterized by the equation:
y=100−100*l^{(-a*Xb)}
where,
y=% dissolution,
x=sampling time,
a=scale parameter which ranges from about 0.0004 to about 0.07,
b=shape parameter which ranges from about 1.48 to about 3.02, and
100=the cumulative percentage of the at least one form of venlafaxine released at time infinity.

101. The composition of claim 83, wherein said at least one form of venlafaxine is venlafaxine hydrochloride and wherein said composition provides an in vitro dissolution profile using the USP type I method at 75 rpm in 1000 ml phosphate buffer pH 6.8 at 37° C. such that between about 0% and about 6.8% venlafaxine hydrochloride is released after about 1 hour, about 0.5% to about 18% is released after about 2 hours, about 3% to about 42% is released after about 4 hours, about 9% to about 63% is released after about 6 hours, about 19% to about 78% is released after about 8 hours, about 34% to about 88% is released after about 10 hours, about 52% to about 94% is released after about 12 hours, and no less than about 100% is released after about 18 hours.

102. The composition of claim 83, wherein said at least one form of venlafaxine is present from about 10 to about 70% by weight of the core dry weight.

103. The composition of claim 83, wherein said at least one form of venlafaxine is present from about 25 to about 60% by weight of the core dry weight.

104. The composition of claim 83, wherein said at least one form of venlafaxine is present at about 55% by weight of the core dry weight.

105. The composition of claim 83, wherein the at least one form of venlafaxine is present from about 0.5 to about 1000 mg.

106. The composition of claim 83, wherein the at least one form of venlafaxine is present from about 5 to about 500 mg.

107. The composition of claim 83, wherein the at least one form of venlafaxine is present from about 100 to about 200 mg.

108. The composition of claim 83, wherein the at least one form of venlafaxine is present at about 150 mg.

109. The composition of claim 83, wherein said at least one form of venlafaxine is a pharmaceutically acceptable salt of venlafaxine.

110. The composition of claim 109, wherein said pharmaceutically acceptable salt of venlafaxine is venlafaxine hydrochloride.

111. The composition of claim 110, wherein said venlafaxine hydrochloride is present at about 150 mg.

112. The composition of claim 83, wherein said active metabolite of venlafaxine is O-desmethylvenlafaxine.

113. The composition of claim 83, wherein said pharmaceutically acceptable salt of an active metabolite of venlafaxine is O-desmethylvenlafaxine succinate.

114. The composition of claim 83, wherein said pharmaceutically acceptable excipient is selected from the group consisting of a gelling agent, a filler, a lubricant and combinations thereof.

115. The composition of claim 114, wherein said gelling agent is present from about 10 to about 80% by weight of the core dry weight.

116. The composition of claim 114, wherein said gelling agent is present from about 10 to about 40% by weight of the core dry weight.

117. The composition of claim 114, wherein said gelling agent is present at about 21% by weight of the core dry weight.

118. The composition of claim 114, wherein gelling agent is selected from the group consisting of hydroxypropylmethylcellulose, hydroxypropylcellulose, polyethylene oxide, polyvinylpyrrolidone, carbomers, carragheen, polyvinylalcohol and any combination thereof.

119. The composition of claim 114, wherein said gelling agent is a mixture of at least two gelling agents.

120. The composition of claim 119, wherein said at least two gelling agents comprise hydroxypropylmethylcellulose at about 13% and polyvinylpyrrolidone at about 8% by weight of the core dry weight.

121. The composition of claim 114, wherein said lubricant is present from about 0.02 to about 5% by weight of the core dry weight.

122. The composition of claim 114, wherein said lubricant is present from about 0.5 to about 2% by weight of the core dry weight.

123. The composition of claim 114, wherein said lubricant is present from about 0.5 to about 1% by weight of the core dry weight.

124. The composition of claim 114, wherein said lubricant is present at about 0.65% by weight of the core dry weight.

125. The composition of claim 114, wherein said lubricant is selected from the group consisting of magnesium stearate, talc, stearic acid, sodium stearyl fumarate, calcium stearate, vegetable oil, silica gel, colloidal silicon dioxide, Compritol 888 ATO and any combination thereof.

126. The composition of claim 114, wherein said lubricant is magnesium stearate.

127. The composition of claim 126, wherein said magnesium stearate comprises about 0.65% by weight of said core dry weight.

128. The composition of claim 114, wherein said filler is present up to about 75% by weight of the core dry weight.

129. The composition of claim 114, wherein said filler is present up to about 50% by weight of the core dry weight.

130. The composition of claim 114, wherein said filler is present up to about 25% by weight of the core dry weight.

131. The composition of claim 114, wherein said filler is present at about 23% by weight of the core dry weight.

132. The composition of claim 114, wherein said at least one filler is selected from the group consisting of lactose monohydrate, anhydrous lactose, mannitol, sorbitol, microcrystalline cellulose, dibasic calcium, calcium sulfate and mixtures thereof.

133. The composition of claim 114, wherein said filler is lactose monohydrate.

134. The composition of claim 114, wherein said filler is Lactose # 315 Spray Dried.

135. The composition of claim 134, wherein said Lactose # 315 Spray Dried is present at about 23% by weight of said core dry weight.

136. The composition of claim 83, wherein said water-insoluble water-permeable film-forming polymer is present from about 20 to about 85% by weight of the coat dry weight.

137. The composition of claim 83, wherein said water-insoluble water-permeable film-forming polymer is present from about 55 to about 62% by weight of the coat dry weight.

138. The composition of claim 83, wherein said water-insoluble water-permeable film-forming polymer is present at about 55% by weight of the coating dry weight.

139. The composition of claim 83, wherein said water-insoluble water-permeable film-forming polymer is selected from the group consisting of ethylcellulose, cellulose acetate, methacrylic acid derivatives, Surelease®, Sureteric®, Acryl-EZE®, and combinations thereof.

140. The composition of claim 83, wherein said water-insoluble water-permeable film-forming polymer is ethylcellulose.

141. The composition of claim 140, wherein said ethylcellulose comprises by weight from about 55 to about 62% by weight of the coat dry weight.

142. The composition of claim 140, wherein said ethylcellulose is present at about 55% by weight of the coat dry weight.

143. The composition of claim 83, wherein said water-soluble polymer or substance is present from about 10 to about 75% by weight of the coating dry weight.

144. The composition of claim 83, wherein said water-soluble polymer or substance is present from about 25 to about 35% by weight of the coating dry weight.

145. The composition of claim 83, wherein said water-soluble polymer or substance is present at about 32% by weight of the coating dry weight.

146. The composition of claim 83, wherein said water-soluble polymer or substance is selected from the group consisting of polyvinylpyrrolidone, polyethyleneglycol, hydroxypropylmethylcellulose, hydrated colloidal silica, sucrose, mannitol, and any combination thereof.

147. The composition of claim 83, wherein water-soluble polymer is polyvinylpyrrolidone.

148. The composition of claim 83, wherein said polyvinylpyrrolidone is present from about 25 to about 35% by weight of the coating dry weight.

149. The composition of claim 83, wherein said polyvinylpyrrolidone is present at about 32% by weight of the coating dry weight.

150. The composition of claim 83, wherein said plasticizer is present from about 3 to about 40% by weight of the coating dry weight.

151. The composition of claim 83, wherein said plasticizer is present from about 10 to about 20% by weight of the coating dry weight.

152. The composition of claim 83, wherein said plasticizer is present from about 13 to about 15% by weight of the coating dry weight.

153. The composition of claim 83, wherein said plasticizer is present at about 13.5% by weight of the coating dry weight.

154. The composition of claim 83, wherein said plasticizer is selected from the group consisting of citrate esters, dibutyl sebacate, dibutyl pthalate, triacetin, castor oil, polyalkyleneglycol, fatty acids, and any combination thereof.

155. The composition of claim 83, wherein the plasticizer is stearic acid.

156. The composition of claim 155, wherein said stearic acid is present from about 13 to about 14% by weight of the coating dry weight.

157. The composition of claim 156, wherein said stearic acid is present at about 13.5% of the coating based on the coating dry weight.

158. The composition of claim 83, wherein the weight proportions of the water-insoluble water-permeable film-forming polymer: water-soluble polymer or substance: plasticizer is about 50-85:10-40:5-20.

159. The composition of claim 83, wherein said a water-insoluble water-permeable film-forming polymer is ethylcellulose, said water-soluble polymer is polyvinylpyrrolidone, and said plasticizer is stearic acid.

160. The composition of claim 159, wherein the weight proportions of ethylcellulose:polyvinylpyrrolidone:stearic acid is about 55-62:26-32: 13-14.

161. The oral dosage form of claim 159, wherein the weight proportions of ethylcellulose:polyvinylpyrrolidone:stearic acid is about 55:32:13.5.

162. The composition of claims 83, when administered to a patient in need thereof provides a similar or diminished incidence of adverse events not influenced by food in comparison to the reference product.

163-244. (canceled)
